SEQUENCE LISTING (44 sequences)

SEQ ID NO: 1 - protein, 1210 aa, Homo sapiens [sequence omitted]

SEQ ID NO: 2 - protein, 335 aa, Artificial Sequence
  example epidermal growth factor receptor (EGFRt) amino acid sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 3 - DNA, 1005 bp, Artificial Sequence
  example nucleotide sequence encoding EGFRt
agaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatg

SEQ ID NO: 4 - protein, 28 aa, Artificial Sequence
  example NGFR signal peptide
MGAGATGRAMDGPRLLLLLLLGVSLGGA

SEQ ID NO: 5 - DNA, 84 bp, Artificial Sequence
  example nucleotide sequence encoding an NGFR signal peptide
atgggagctggtgctaccggcagagctatggatggacctagactgctgctcctgctgctgctcggagtttctcttggcggagcc

SEQ ID NO: 6 - protein, 22 aa, Artificial Sequence
  example GMS SFR alpha signal peptide
MLLLVTSLLLCELPHPAFLLIP

SEQ ID NO: 7 - DNA, 66 bp, Artificial Sequence
  example GMS SFR alpha signal peptide
atgctgctgctggtcacctctctgctgctgtgcgaactgccccatcctgcctttctgctgatcccc

SEQ ID NO: 8 - DNA, 24 bp, Artificial Sequence
  example nucleotide sequence encoding a cytoplasmic tail
aagcggtggaaccggggcatcctg

SEQ ID NO: 9 - DNA, 24 bp, Artificial Sequence
  example nucleotide sequence encoding a cytoplasmic tail
cggcggagacacatcgtgcggaag

SEQ ID NO: 10 - protein, 23 aa, Artificial Sequence
  epidermal growth factor receptor (EGFR) transmembrane domain
IATGMVGALLLLLVVALGIGLFM

SEQ ID NO: 11 - DNA, 69 bp, Artificial Sequence
  nucleotide sequence encoding an EGFR transmembrane domain
atcgccaccggcatggttggagccctgctgcttctgctggtggtggcccttggaatcggcctgtttatg

SEQ ID NO: 12 - protein, 22 aa, Artificial Sequence
  NGFR transmembrane domain
LIPVYCSILAAVVVGLVAYIAF

SEQ ID NO: 13 - DNA, 66 bp, Artificial Sequence
  nucleotide sequence encoding an NGFR transmembrane domain
ctgatccccgtgtactgtagcatcctggccgccgtggttgtgggactcgtggcctatatcgccttc

SEQ ID NO: 14 - protein, 24 aa, Artificial Sequence
  recycling signal sequence
YQPLSQIKRLLSDSFLFDNPVYAS

SEQ ID NO: 15 - DNA, 72 bp, Artificial Sequence
  nucleotide sequence encoding a recycling signal
tatcagcctctgagccagatcaagcggctgctgagcgactccttcctgttcgacaaccccgtgtacgctagc

SEQ ID NO: 16 - DNA, 1089 bp, Artificial Sequence
  nucleotide sequence encoding modified EGFRt (with NGFR signal peptide)
atgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatg

SEQ ID NO: 17 - protein, 363 aa, Artificial Sequence
  modified EGFRt (with NGFR signal peptide)
MGAGATGRAMDGPRLLLLLLLGVSLGGARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 18 - DNA, 1038 bp, Artificial Sequence
  nucleotide sequence encoding modified EGFRt (with NGFR cytoplasmic domain)
agaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgaagcggtggaaccggggcatcctggctagctga

SEQ ID NO: 19 - protein, 345 aa, Artificial Sequence
  modified EGFRt (with NGFR cytoplasmic domain)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMKRWNRGILAS

SEQ ID NO: 20 - DNA, 1038 bp, Artificial Sequence
  nucleotide sequence encoding modified EGFRt (with EGFR cytoplasmic domain)
agaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgcggcggagacacatcgtgcggaaggctagctga

SEQ ID NO: 21 - protein, 345 aa, Artificial Sequence
  modified EGFRt (with EGFR cytoplasmic domain)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKAS

SEQ ID NO: 22 - DNA, 1122 bp, Artificial Sequence
  nucleotide sequence encoding modified EGFRt (with NGFR signal peptide and NGFR cytoplasmic domain)
atgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgaagcggtggaaccggggcatcctggctagctga

SEQ ID NO: 23 - protein, 373 aa, Artificial Sequence
  modified EGFRt (with NGFR signal peptide and NGFR cytoplasmic domain)
MGAGATGRAMDGPRLLLLLLLGVSLGGARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMKRWNRGILAS

SEQ ID NO: 24 - DNA, 1122 bp, Artificial Sequence
  nucleotide sequence encoding modified EGFRt (with NGFR signal peptide and EGFR cytoplasmic domain)
atgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgcggcggagacacatcgtgcggaaggctagctga

SEQ ID NO: 25 - protein, 373 aa, Artificial Sequence
  modified EGFRt (with NGFR signal peptide and EGFR cytoplasmic domain)
MGAGATGRAMDGPRLLLLLLLGVSLGGARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKAS

SEQ ID NO: 26 - DNA, 1074 bp, Artificial Sequence
  comparative nucleotide sequence encoding an EGFRt
atgctgctgctggtcacctctctgctgctgtgcgaactgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgtag

SEQ ID NO: 27 - protein, 357 aa, Artificial Sequence
  comparative EGFRt sequence
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 28 - DNA, 1146 bp, Artificial Sequence
  nucleotide sequence encoding modified EGFRt (with GMS SFR alpha signal peptide; and NGFR transmembrane and cytoplasmic domain)
atgctgctgctggtcacctctctgctgctgtgcgaactgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctggcggcggaggatctggcggaggtggaagcggaggcggtggatctctgatccccgtgtactgtagcatcctggccgccgtggttgtgggactcgtggcctatatcgccttcaagcggtggaaccggggcatcctggctagctga

SEQ ID NO: 29 - protein, 381 aa, Artificial Sequence
  modified EGFRt (with GMS SFR alpha signal peptide; and NGFR transmembrane and cytoplasmic domain)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGGGSGGGGSGGGGSLIPVYCSILAAVVVGLVAYIAFKRWNRGILAS

SEQ ID NO: 30 - DNA, 1194 bp, Artificial Sequence
  nucleotide sequence encoding modified EGFRt (with NGFR signal peptide, NGFR cytoplasmic domain and recycling signal)
atgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgaagcggtggaaccggggcatcctggctagctatcagcctctgagccagatcaagcggctgctgagcgactccttcctgttcgacaaccccgtgtacgctagctga

SEQ ID NO: 31 - protein, 397 aa, Artificial Sequence
  modified EGFRt (with NGFR signal peptide, NGFR cytoplasmic domain and recycling signal)
MGAGATGRAMDGPRLLLLLLLGVSLGGARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMKRWNRGILASYQPLSQIKRLLSDSFLFDNPVYAS

SEQ ID NO: 32 - DNA, 1194 bp, Artificial Sequence
  nucleotide sequence encoding modified EGFRt (with NGFR signal peptide; and EGFR cytoplasmic domain and recycling signal)
atgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgcggcggagacacatcgtgcggaaggctagctatcagcccctgagccagatcaagagactgctgagcgactccttcctgttcgacaaccccgtgtacgctagctga

SEQ ID NO: 33 - protein, 397 aa, Artificial Sequence
  modified EGFRt (with NGFR signal peptide; and EGFR cytoplasmic domain and recycling signal)
MGAGATGRAMDGPRLLLLLLLGVSLGGARKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKASYQPLSQIKRLLSDSFLFDNPVYAS

SEQ ID NO: 34 - DNA, 1218 bp, Artificial Sequence
  nucleotide sequence encoding modified EGFRt (with GMS SFR alpha signal peptide; and NGFR transmembrane and cytoplasmic domain and recycling signal)
atgctgctgctggtcacctctctgctgctgtgcgaactgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctggcggcggaggatctggcggaggtggaagcggaggcggtggatctctgatccccgtgtactgtagcatcctggccgccgtggttgtgggactcgtggcctatatcgccttcaagcggtggaaccggggcatcctggctagctatcagcctctgagccagatcaagcggctgctgagcgactccttcctgttcgacaaccctgtgtacgctagctga

SEQ ID NO: 35 - protein, 405 aa, Artificial Sequence
  modified EGFRt (with GMS SFR alpha signal peptide; and NGFR transmembrane and cytoplasmic domain and recycling signal)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGGGSGGGGSGGGGSLIPVYCSILAAVVVGLVAYIAFKRWNRGILASYQPLSQIKRLLSDSFLFDNPVYAS

SEQ ID NO: 36 - DNA, 3477 bp, Artificial Sequence
  unmodified EGFRt
tttacatgtgctcttaattacagcagaaccggtctgacctcttctcttcctcccacagatcgaggacgagagaaacctgcacgaggacttcgtgttcatgaagaccatccagcggtgcaacaccggcgagagaagtctgagcctgctgaactgcgaggaaatcaagagccagttcgagggcttcgtgaaggacatcatgctgaacaaagaggaaacgaagaaagaaaactccttcgagatgcagaagggcgaccagaatcctcagatcgccgctcacgtgatcagcgaggccagcagcaagacaacaagcgtgctgcagtgggccgagaagggctactacaccatgagcaacaacctggtcaccctggaaaacggcaagcagctgacagtgaagcggcagggcctgtactacatctacgcccaagtgaccttctgcagcaacagagaggccagctctcaggccccttttatcgccagcctgtgcctgaagtcccctggcagattcgagcggattctgctgagagccgccaacacacacagcagcgccaaaccttgtggccagcagtctattcacctcggcggagtgtttgagctgcagcctggcgcaagcgtgttcgtgaatgtgacagaccctagccaggtgtcccacggcaccggctttacatctttcggactgctgaagctgtgaacagtgtcaccttgcaggctgtggtggagctgacgctgggagtcttcataatacagcacagcggttaagcccaccccctgttaactgcctatttataaccctagggaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgctgctgctggtcacctctctgctgctgtgcgaactgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgtagcctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacgatgtctccattgtttccagagtgaacttgtaattatcttgttatttattttttgaataataaagacctcttaacattacgcgcttaacattatcgttgttgtttgagtacctaaagctcccagccaggttggggaaagaggaagcatttggagggaattttcccaacctttgtgatgttttcataaactttgttctcaagctacttacatt

SEQ ID NO: 37 - DNA, 3525 bp, Artificial Sequence
  modified EGFRt with NGFR signal peptide and NGFR cytoplasmic domain
tttacatgtgctcttaattacagcagaaccggtctgacctcttctcttcctcccacagatcgaggacgagagaaacctgcacgaggacttcgtgttcatgaagaccatccagcggtgcaacaccggcgagagaagtctgagcctgctgaactgcgaggaaatcaagagccagttcgagggcttcgtgaaggacatcatgctgaacaaagaggaaacgaagaaagaaaactccttcgagatgcagaagggcgaccagaatcctcagatcgccgctcacgtgatcagcgaggccagcagcaagacaacaagcgtgctgcagtgggccgagaagggctactacaccatgagcaacaacctggtcaccctggaaaacggcaagcagctgacagtgaagcggcagggcctgtactacatctacgcccaagtgaccttctgcagcaacagagaggccagctctcaggccccttttatcgccagcctgtgcctgaagtcccctggcagattcgagcggattctgctgagagccgccaacacacacagcagcgccaaaccttgtggccagcagtctattcacctcggcggagtgtttgagctgcagcctggcgcaagcgtgttcgtgaatgtgacagaccctagccaggtgtcccacggcaccggctttacatctttcggactgctgaagctgtgaacagtgtcaccttgcaggctgtggtggagctgacgctgggagtcttcataatacagcacagcggttaagcccaccccctgttaactgcctatttataaccctagggaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgaagcggtggaaccggggcatcctggctagctgacctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacgatgtctccattgtttccagagtgaacttgtaattatcttgttatttattttttgaataataaagacctcttaacattacgcgcttaacattatcgttgttgtttgagtacctaaagctcccagccaggttggggaaagaggaagcatttggagggaattttcccaacctttgtgatgttttcataaactttgttctcaagctacttacatt

SEQ ID NO: 38 - DNA, 3525 bp, Artificial Sequence
  modified EGFRt with NGFR signal peptide and EGFR cytoplasmic domain
tttacatgtgctcttaattacagcagaaccggtctgacctcttctcttcctcccacagatcgaggacgagagaaacctgcacgaggacttcgtgttcatgaagaccatccagcggtgcaacaccggcgagagaagtctgagcctgctgaactgcgaggaaatcaagagccagttcgagggcttcgtgaaggacatcatgctgaacaaagaggaaacgaagaaagaaaactccttcgagatgcagaagggcgaccagaatcctcagatcgccgctcacgtgatcagcgaggccagcagcaagacaacaagcgtgctgcagtgggccgagaagggctactacaccatgagcaacaacctggtcaccctggaaaacggcaagcagctgacagtgaagcggcagggcctgtactacatctacgcccaagtgaccttctgcagcaacagagaggccagctctcaggccccttttatcgccagcctgtgcctgaagtcccctggcagattcgagcggattctgctgagagccgccaacacacacagcagcgccaaaccttgtggccagcagtctattcacctcggcggagtgtttgagctgcagcctggcgcaagcgtgttcgtgaatgtgacagaccctagccaggtgtcccacggcaccggctttacatctttcggactgctgaagctgtgaacagtgtcaccttgcaggctgtggtggagctgacgctgggagtcttcataatacagcacagcggttaagcccaccccctgttaactgcctatttataaccctagggaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggagctggtgctaccggcagagctatggatggacctagactgctgctcctgctgctgctcggagtttctcttggcggagccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatgcggcggagacacatcgtgcggaaggctagctgacctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacgatgtctccattgtttccagagtgaacttgtaattatcttgttatttattttttgaataataaagacctcttaacattacgcgcttaacattatcgttgttgtttgagtacctaaagctcccagccaggttggggaaagaggaagcatttggagggaattttcccaacctttgtgatgttttcataaactttgttctcaagctacttacatt

SEQ ID NO: 39 - protein, 8 aa, Artificial Sequence
  cytoplasmic tail sequence
KRWNRGIL

SEQ ID NO: 40 - protein, 8 aa, Artificial Sequence
  cytoplasmic tail sequence
RRRHIVRK

SEQ ID NO: 41 - protein, 10 aa, Artificial Sequence
  cytoplasmic tail sequence
KRWNRGILAS

SEQ ID NO: 42 - protein, 10 aa, Artificial Sequence
  cytoplasmic tail sequence
RRRHIVRKAS

SEQ ID NO: 43 - DNA, 30 bp, Artificial Sequence
  example nucleotide sequence encoding a cytoplasmic tail
aagcggtggaaccggggcatcctggctagc

SEQ ID NO: 44 - DNA, 30 bp, Artificial Sequence
  example nucleotide sequence encoding a cytoplasmic tail
cggcggagacacatcgtgcggaaggctagc

(19) United States
(12) Patent Application Publication
(10) Pub. No.: US 2023/0174622 A1
(43) Pub. Date: Jun. 8, 2023

(54) EPIDERMAL GROWTH FACTOR RECEPTOR

(71) Applicants: Ospedale San Raffaele S.R.L., Milan (IT); Fondazione Telethon, Rome (IT)

(72) Inventors: Luigi Naldini, Milan (IT); Pietro Genovese, Milan (IT); Valentina Vavassori, Milan (IT)

(21) Appl. No.: 17/998,447

(22) PCT Filed: May 14, 2021
(86) PCT No.: PCT/EP2021/062873

§ 371 Date: Nov. 10, 2022

(30) Foreign Application Priority Data
May 14, 2020 (GB) 2007169.2

(51) Int. Cl. C07K 14/71; C12N 15/86; C07K 16/28; A61P 37/06

(52) U.S. Cl. CPC C07K 14/71; C12N 15/86; C07K 16/2863; A61P 37/06; C07K 2319/03

(57) ABSTRACT

A polynucleotide comprising a nucleotide sequence encoding an epidermal growth factor receptor (EGFR) extracellular epitope operably linked to: (a) a NGFR or GMS SFR alpha signal peptide; (b) a EGFR or NGFR transmembrane domain; and/or (c) a NGFR or EGFR cytosplasmic tail.

### FIELD OF THE INVENTION

The present invention relates to modified epidermal growth factor receptors (EGFRs). In particular, the invention relates to the use of the modified EGFRs in selecting, depleting and tracking populations of cells that have been engineered to express the modified EGFRs.

### BACKGROUND TO THE INVENTION

Administration of genetically engineered cells, such as adoptive transfer of engineered T cells or transplantation of engineered hematopoietic stem or progenitor cells are powerful tools in therapy, for example for treating infectious, neoplastic and inherited diseases.

Selection of engineered cells before administration to subjects, thus reducing or eliminating cells lacking therapeutic efficacy, is required in some cases. In addition, tracking engineered cells after infusion and depleting or eliminating them in case of toxicity are advantageous capabilities of genetically manipulated cell products.

Several strategies have been used to select genetically engineered cells in vitro or to track and deplete genetically engineered cells in vivo. In vitro selection through the use of xenogenic enzymes has been employed, however such approaches suffer from issues with immunogenicity. Human cell surface proteins, such as ΔNGFR, CD34, CD19, CD20 and CD4, and CD90, have also been used as surrogate markers for the identification of ex vivo genetically modified cells. However, none of these candidates is ideal because they are not unique to the engineered hematopoietic cells. Indeed, they are expressed in a variety of tissues comprising the blood compartment, making the tracking of specific modified cells challenging and exacerbating the toxicity of treatment in case of depletion.

A functionally inert truncated version of human epidermal growth factor receptor (EGFRt) has been proposed as a candidate for selection, tracking and depletion of modified cells (Wang et al. (2011) Blood 118(5): 1255-1263). EGFR is not expressed by cells of the hematopoietic systems. To generate EGFRt, the wild-type receptor was rendered incapable of binding ligands and of signalling activity by removal of two extracellular domains and of its cytoplasmic tail, although an intact cetuximab binding site was retained.

EGFRt must be expressed at high levels on the cell surface for optimal functionality, such as to allow complete elimination of transplanted cells and to avoid the use of high doses of monoclonal antibody, which could result in deleterious side effects. However, expression of previous EGFRt constructs is low in several contexts, such as when using conventional transcriptional promoters.

This issue is particularly relevant with cells transduced with bi-directional or bi-cistronic vectors (i.e. expressing two gene products from the same transcript, such as through an internal ribosomal entry sequence, IRES) or when the EGFRt is fused to another therapeutic product (i.e. through a self-cleaving peptide) or where EGFRt expression is linked by gene editing to low-expressed target loci. This represents a significant safety concern when cells need to be selected, tracked in vivo and depleted in case of adverse events.

Accordingly, there remains a significant need in the art for means of selecting, depleting and tracking genetically engineered cells, particularly in contexts where an introduced construct may be under the control of a weak expression control system.

### SUMMARY OF THE INVENTION

The present inventors have developed an engineered epidermal growth factor receptor, which exhibits increased stability and expression on the surface of cells even when its expression is driven by a weak promoter.

The inventors have optimised EGFRt in order to improve translation and cell surface stability to produce enhanced EGFRt (eEGFRt) through utilisation of: (i) atypical signal peptides to efficiently drive protein translation to the endoplasmic reticulum; (ii) an engineered cytoplasmic tail that stabilises and increases cell surface recycling of the EGFR protein; and/or (iii) optimisation of the open reading frame sequence to increase protein translation. By applying these modifications, the inventors have demonstrated improved utility of the eEGFRt marker on genetically modified low-expressing cells by allowing recovery of the genetically modified cells in vitro, and their depletion in vitro and in vivo, by the administration of a lower dose of monoclonal antibody. This improvement increases the safety profile of engineered cells in the case of an adverse event, by eliminating low-expressing cells and reducing the risk of unwanted effects of the depleting antibody that is administered.

In one aspect the invention provides a polynucleotide comprising a nucleotide sequence encoding an epidermal growth factor receptor (EGFR) extracellular epitope operably linked to:
(a) a NGFR or GMS SFR alpha signal peptide;
(b) a EGFR or NGFR transmembrane domain; and/or
(C) a NGFR or EGFR cytosplasmic tail,

and optionally a recycling signal.

In some embodiments the cytoplasmic tail comprises an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In some embodiments the EGFR extracellular epitope comprises one or more EGFR extracellular domains or parts thereof. In some embodiments the EGFR extracellular epitope comprises a EGFR Domain III or part thereof. In some embodiments the EGFR extracellular epitope comprises a EGFR Domain IV or part thereof. In some embodiments the EGFR extracellular epitope comprises a EGFR Domain III and a EGFR Domain IV or parts thereof.

In some embodiments the EGFR extracellular epitope is a truncated epidermal growth factor receptor (EGFRt).

In another aspect the invention provides a polynucleotide comprising a nucleotide sequence encoding a truncated epidermal growth factor receptor (EGFRt), wherein the polynucleotide further comprises:
(a) a nucleotide sequence encoding a NGFR signal peptide; and/or
(b) a nucleotide sequence encoding a polypeptide comprising an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In some embodiments:
(a) the NGFR signal peptide comprises an amino acid sequence having at least 70% identity to SEQ ID NO: 4; and/or
(b) the nucleotide sequence encoding the NGFR signal peptide has at least 70% identity to SEQ ID NO: 5.

In some embodiments the EGFRt comprises a EGFR Domain III and a EGFR Domain IV. In some embodiments the EGFRt further comprises a EGFR transmembrane domain or a NGFR transmembrane domain, preferably a EGFR transmembrane domain. In some embodiments the EGFRt does not comprise a EGFR Domain I, a EGFR Domain II, a EGFR Juxtamembrane Domain or a EGFR Tyrosine Kinase Domain.

In some embodiments the EGFRt comprises an amino acid sequence having at least 70% identity to SEQ ID NO: 2.

In some embodiments the nucleotide sequence encoding the EGFRt has at least 70% identity to SEQ ID NO: 3.

In some embodiments the polynucleotide:
(a) encodes an amino acid sequence having at least 70% identity to SEQ ID NO: 17; and/or
(b) comprises a nucleotide sequence having at least 70% identity to SEQ ID NO: 16.

In some embodiments the polynucleotide:
(a) encodes an amino acid sequence having at least 70% identity to SEQ ID NO: 19 or 21; and/or
(b) comprises a nucleotide sequence having at least 70% identity to SEQ ID NO: 18 or 20.

In some embodiments the polynucleotide:
(a) encodes an amino acid sequence having at least 70% identity to SEQ ID NO: 23 or 25; and/or
(b) comprises a nucleotide sequence having at least 70% identity to SEQ ID NO: 22 or 24.

In some embodiments the nucleotide sequence encoding the EGFRt is operably linked to a weak promoter.

In some embodiments the polynucleotide further comprises a transgene.

In some embodiments the polynucleotide, for example comprising the transgene and/or EGFRt-encoding sequence, is for insertion by gene editing.

In some embodiments the transgene encodes a chimeric antigen receptor.

In another aspect the invention provides an EGFRt protein encoded by the polynucleotide of the invention.

In another aspect the invention provides a viral vector comprising the polynucleotide of the invention.

In some embodiments the viral vector is a lentiviral vector, adeno-associated viral (AAV) vector or adenoviral vector.

In another aspect the invention provides a cell comprising the polynucleotide or the viral vector of the invention.

In another aspect the invention provides the polynucleotide, viral vector or cell of the invention for use in therapy.

In another aspect the invention provides use of the polynucleotide, viral vector or cell of the invention for the manufacture of a medicament.

In another aspect the invention provides a method of selecting transduced cells comprising the steps:
(a) transducing a population of cells with the polynucleotide or viral vector of the invention;
(b) contacting the transduced cell population with an EGFRt-binding agent; and
(c) selecting the cells bound to the EGFRt-binding agent.

In another aspect the invention provides a method of depleting transduced cells comprising the steps:
(a) transducing a population of cells with the polynucleotide or viral vector of the invention; and
(b) contacting the transduced cell population with an EGFRt-binding agent.

In some embodiments the EGFRt-binding agent is operably linked to a depletion agent. In some embodiments the cell population of step (b) is contacted with a depletion agent that binds to the EGFRt-binding agent. In some embodiments the binding of the EGFRt-binding agent to EGFRt expressed on the surface of a cell causes death of the cell. In some embodiments the depletion agent kills a cell to which the EGFRt-binding agent is bound.

In another aspect the invention provides a method of tracking transduced cells comprising the steps:
(a) transducing a population of cells with the polynucleotide or viral vector of the invention;
(b) contacting the transduced cell population with an EGFRt-binding agent, wherein the EGFRt-binding agent is operably linked to a detectable label; and
(c) detecting the cells bound to the EGFRt-binding agent.

In some embodiments the method is an in vitro or ex vivo method.

In some embodiments the EGFRt-binding agent is an antibody. In some embodiments the EGFRt-binding agent is cetuximab

In some embodiments the depletion agent comprises a toxin. In some embodiments the depletion agent comprises saporin.

In another aspect the invention provides a method of treatment comprising the method of selecting transduced cells, the method of depleting transduced cells and/or the method of tracking transduced cells of the invention.

### DESCRIPTION OF THE DRAWINGS

FIG. 1

EGFRt modifications improved protein surface expression after a CD40LG gene editing procedure, allowing in vitro enrichment and in vitro/in vivo depletion of engineered cells. (A) Schematic representation of three corrective donor templates used to edit CD4+ T lymphocytes. Inserted within intron 1 of CD40LG locus was a corrective donor template with 500 bp homology arms, composed by a splice acceptor (SA) followed by CD40LG cDNA from exon 2 to exon 5 with its endogenous 3′UTR and polyA. An IRES sequence followed by either the EGFRt gene (i) or the eEGFRt 1 gene (ii) or the eEGFRt 2 gene (iii) was cloned between the cDNA and 3′UTR sequences. When editing occurs, the CD40LG endogenous promoter drives the expression of both the CD40LG gene and the EGFRt/modified EGFRt genes. (B) Representative Flow Cytometry dot plots showing EGFR gene expression in edited CD4+ non-activated T cells with relative Mean Fluorescence Intensities (MFI). (C) Histogram plot showing percentages of eEGFRt+ edited cells before immunomagnetic enrichment (eEGFRt 1/eEGFRt 2 bars) and after immunomagnetic enrichment (eEGFRt 1+/eEGFR 2+ bars). eEGFR 1− and eEGFR 2− bars show the percentage of eEGFRt+ cells retained in the negative fraction after the selection procedure. (D) Histogram plot depicting the percentage of eEGFR+ modified cells after treatment with the assembled immunotoxin (black bar), antibody only (red bar) or toxin only (green bar) in vitro. (E) Time course of the percentage of eEGFRt+ T cells retrieved in the peripheral blood (PB) of xenotransplanted NSG mice treated (red line) or not (blue line) with Cetuximab intraperitoneal injections. The percentage of eEGFRt+ cells was assessed both by flow cytometry analysis (i) and by digital droplet PCR molecular assay (ii).

FIG. 2

EGFRt modifications improve protein surface expression after transduction with bi-directional lentiviral vectors. (A) Schematic representation of bi-directional lentiviral vectors expressing in sense the GFP reporter gene under the control of a hPGK promoter and in antisense either the tEGFR (FIG. 2Ai) or the modified EGFRt (eEGFRt 1—FIG. 2Aii) driven by a minimal CMV promoter. (B) Representative flow cytometry dot plots showing EGFR gene expression in transduced T cells with relative Mean Fluorescence Intensities (MFI).

FIG. 3

EGFRt modifications with recycling signals did not further improve EGFR protein expression after a gene editing procedure. Representative flow cytometry dot plots showing EGFRt gene expression in edited CD4+ non-activated (basal level) or activated (stimulation) T cells.

FIG. 4

EGFRt modifications without recycling signals improved EGFR surface expression after a gene editing procedure. Representative flow cytometry dot plots showing a time course of EGFRt gene expression in edited CD4+ non-activated (basal level) T cells with relative Mean Fluorescence Intensities (MFI).

FIG. 5

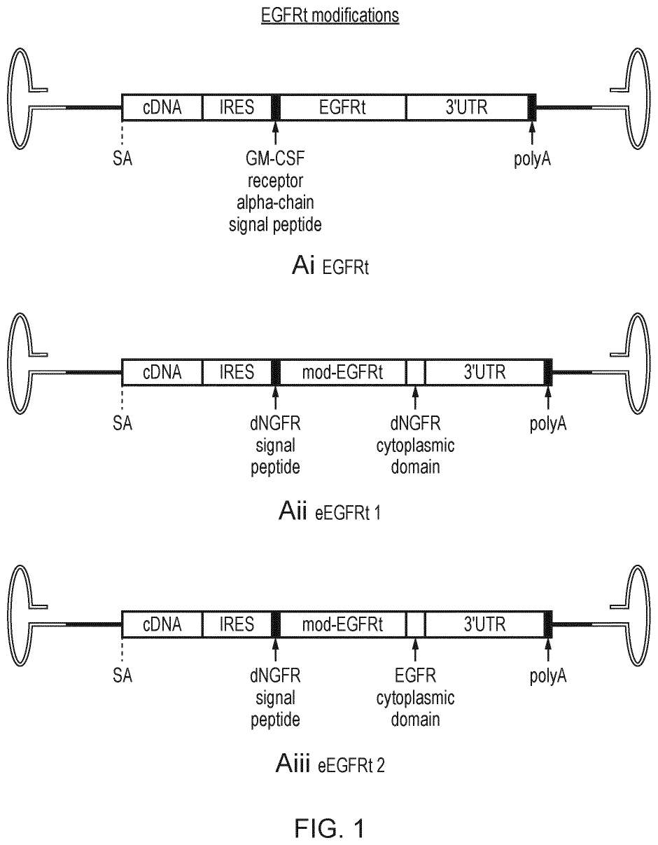

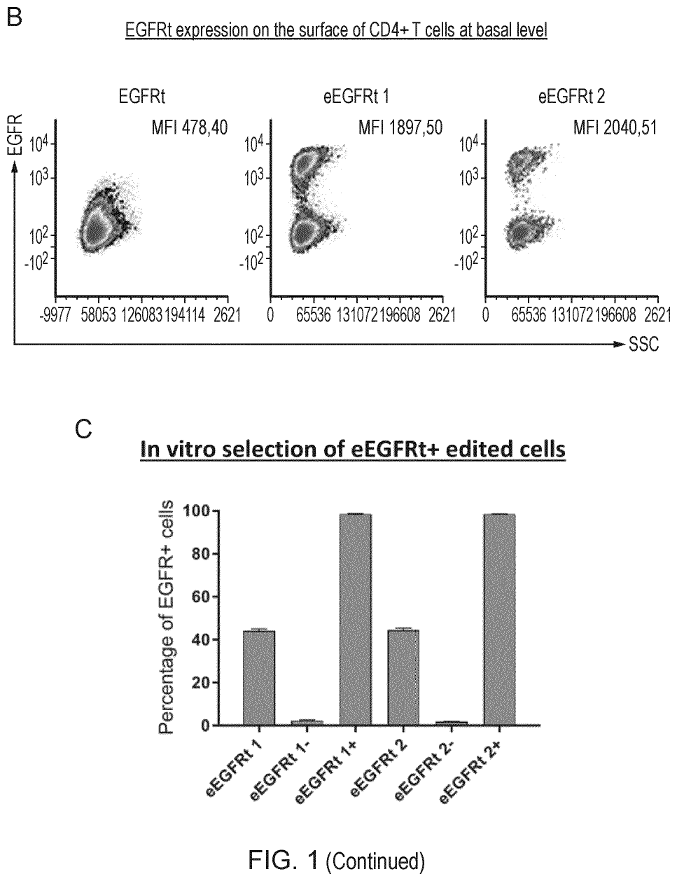

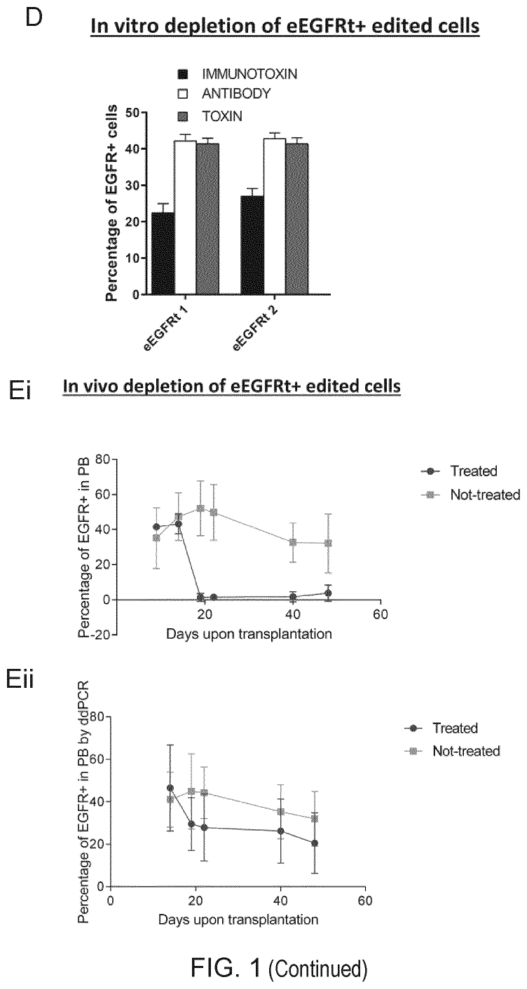

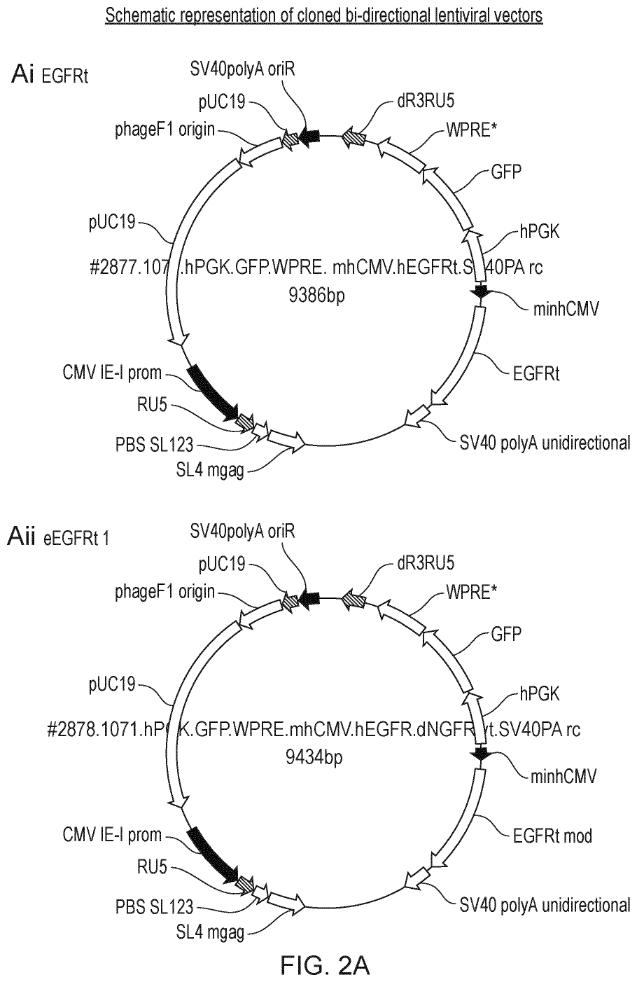

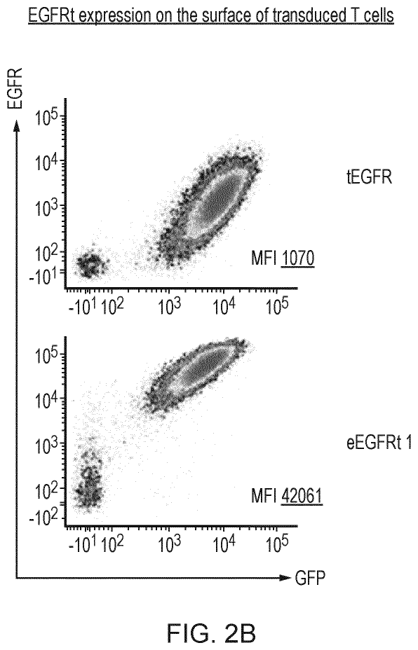

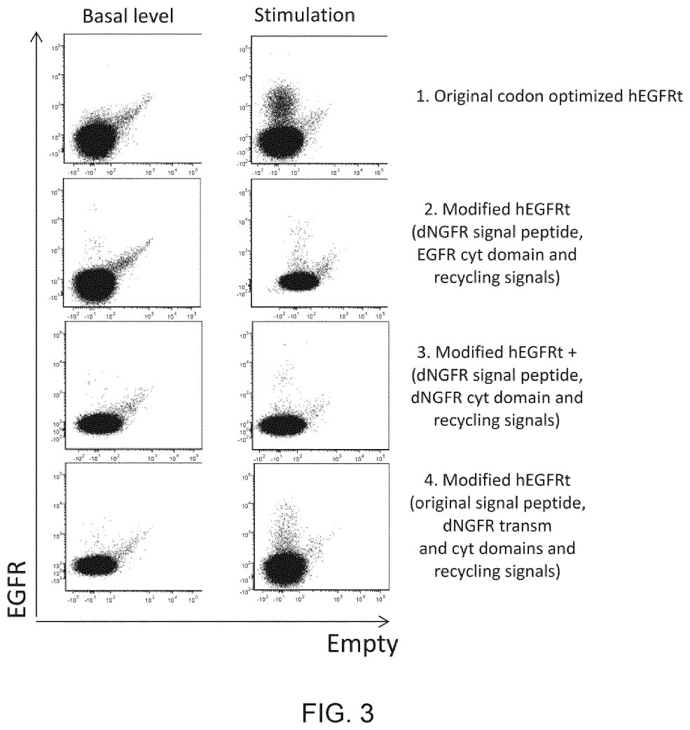

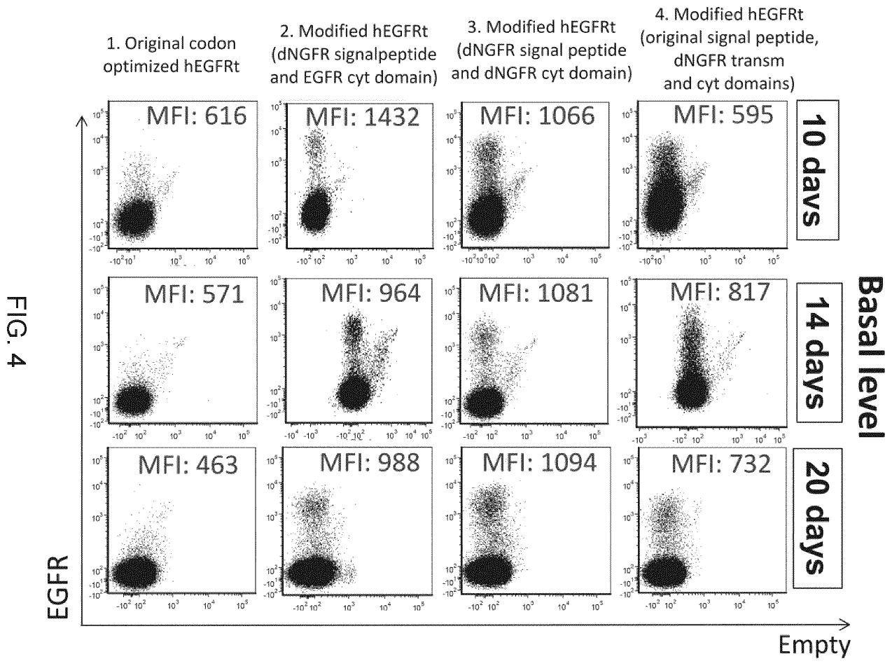

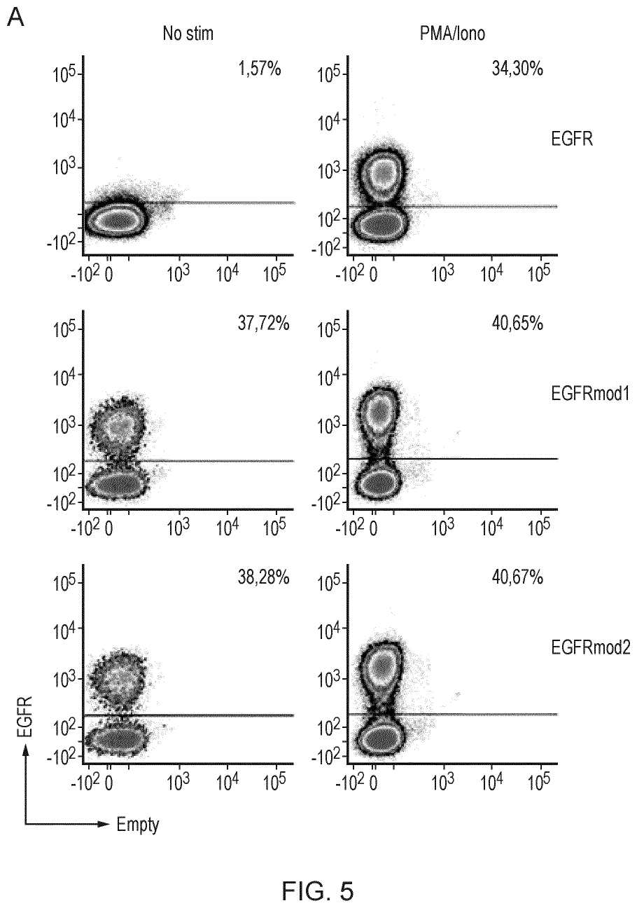

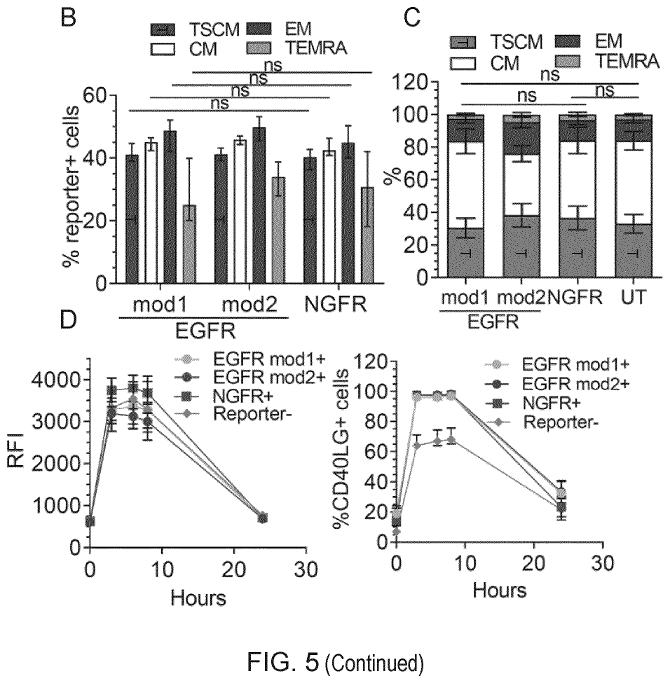

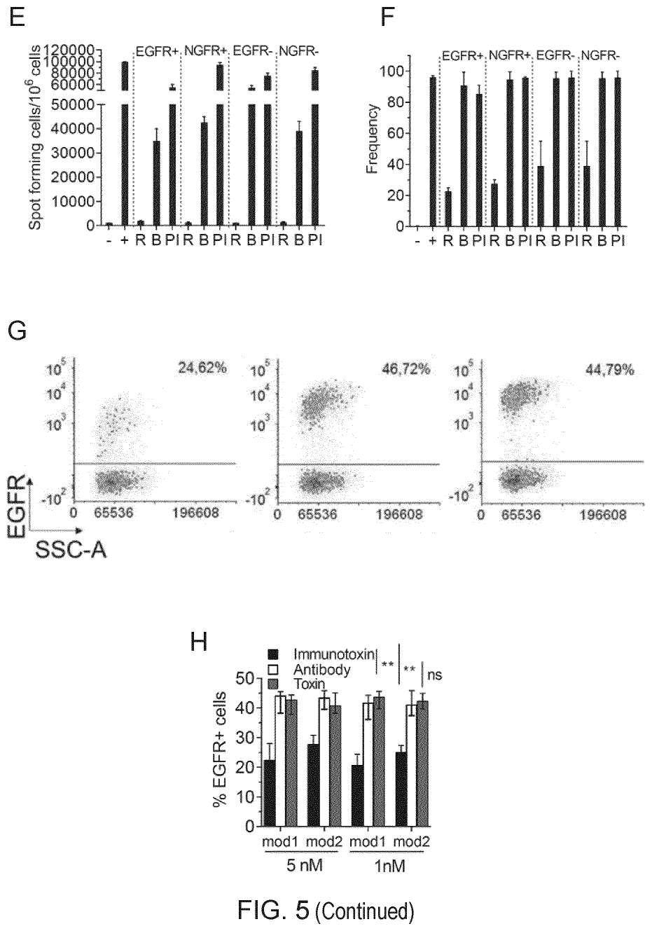

Edited T cells can be specifically depleted by exploiting the clinically compliant selector EGFRt. (A) Representative plots showing EGFRt expression in bulk edited CD4+ T cells derived from male HD at 0 and 8 hr after Pma/lonomycin stimulation. Cells were edited with the three constructs depicted in FIG. 1A. (B) Percentage of Reporter+ cells within T cell subpopulations at 17 days after CD40LG editing in male HD derived CD4+ T cells, measured by FACS analysis. Cells were edited with donor templates carrying NGFR (n=7), eEGFRt 1 (also referred to as EGFRmod1; n=7) or eEGFRt 2 (also referred to as EGFRmod2; n=4). Paired Wilcoxon's test. EGFRmod2 not included in the analysis because n=4. (C) Population composition in male HD derived UT T cells (n=14) or bulk edited T cells from (B). LME model, followed by post-hoc analysis. EGFRmod2 not included in the analysis because n=4. Mean±SEM. (D) Time course of CD40L surface expression after PMA/lonomycin stimulation measured by RFI (normalized to Reporter− cells; left panel) and percentage (right panel) on edited or unedited T cells from (B) (n=4 for each group, except for Reporter− n=12). (E) IgG+ secreting B cells, evaluated by ELISPOT assay. B cells were isolated from PB of HD and co-cultured with male HD sorted NGFR/EGFR+, NGFR/EGFR− and UT T cells, resting (R) or stimulated with beads (B) or PMA/lonomycin (PI). B cells cultured alone (−) or in presence of sCD40L (+) were used as negative and positive controls, respectively (n=2 for each group). (F) Analysis of B cell proliferative capacity by Cell Trace dilution assay in allogeneic sorted B cells isolated from PB of HD and co-cultured with male HD T cells from (E). B cells cultured alone (−) or in presence of sCD40L (+) were used as negative and positive controls, respectively (n=2 for each group). (G) Representative plots showing hEGFRt expression in bulk edited CD4+ T cells derived from male HD at 3 days after treatment with immunotoxin (left), antibody (middle) or toxin (right). (H) Histograms showing percentage of hEGFRt+ T cells at 3 days after treatment with 5 nM or 1 nM of immunotoxin, antibody or toxin, measured by FACS Analysis. Friedman test with Dunn's multiple comparisons. Different dose-conditions were used as a unified group for statistical analysis.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Chimeric Selector

The inventors have developed an engineered cell surface protein that may be used in selection, depletion and tracking of cells on which it is expressed. The protein, which may be referred to herein as a “chimeric selector”, comprises an extracellular epitope of an epidermal growth factor receptor (EGFR), and exhibits increased stability and expression on the surface of cells even when its expression is driven by a weak promoter. The EGFR extracellular epitope may be, for example, selectively bound by an anti-EGFR antibody, such as cetuximab.

In one aspect the invention provides a polynucleotide comprising a nucleotide sequence encoding an epidermal growth factor receptor (EGFR) extracellular epitope operably linked to:
(a) a NGFR or GMS SFR alpha signal peptide;
(b) a EGFR or NGFR transmembrane domain; and/or
(c) a NGFR or EGFR cytosplasmic tail,

and optionally a recycling signal.

In some embodiments the cytoplasmic tail comprises an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In some embodiments the EGFR extracellular epitope comprises one or more EGFR extracellular domains or parts thereof. In some embodiments the EGFR extracellular epitope comprises a EGFR Domain III or part thereof. In some embodiments the EGFR extracellular epitope comprises a EGFR Domain IV or part thereof. In some embodiments the EGFR extracellular epitope comprises a EGFR Domain III and a EGFR Domain IV or parts thereof.

In some embodiments the EGFR extracellular epitope is a truncated epidermal growth factor receptor (EGFRt).

Epidermal Growth Factor Receptor (EGFR)

Epidermal growth factor receptor (EGFR), which may also be known as ErbB1 and HER1, is a cell-surface receptor for members of the epidermal growth factor family of extracellular ligands.

An example sequence of human EGFR is:

(SEQ ID NO: 1)
MRPSGTAGAALLALLAALCPASRALEEKKVCQGTSNKLTQLGTFEDHFL
SLQRMFNNCEVVLGNLEITYVQRNYDLSFLKTIQEVAGYVLIALNTVER
IPLENLQIIRGNMYYENSYALAVLSNYDANKTGLKELPMRNLQEILHGA
VRFSNNPALCNVESIQWRDIVSSDFLSNMSMDFQNHLGSCQKCDPSCPN
GSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKK
CPRNYVVTDHGSCVRACGADSYEMEEDGVRKCKKCEGPCRKVCNGIGIG
EFKDSLSINATNIKHFKNCTSISGDLHILPVAFRGDSFTHTPPLDPQEL
DILKTVKEITGFLLIQAWPENRTDLHAFENLEIIRGRTKQHGQFSLAVV
SLNITSLGLRSLKEISDGDVIISGNKNLCYANTINWKKLFGTSGQKTKI
ISNRGENSCKATGQVCHALCSPEGCWGPEPRDCVSCRNVSRGRECVDKC
NLLEGEPREFVENSECIQCHPECLPQAMNITCTGRGPDNCIQCAHYIDG
PHCVKTCPAGVMGENNTLVWKYADAGHVCHLCHPNCTYGCTGPGLEGCP
TNGPKIPSIATGMVGALLLLLVVALGIGLFMRRRHIVRKRTLRRLLQER
ELVEPLTPSGEAPNQALLRILKETEFKKIKVLGSGAFGTVYKGLWIPEG
EKVKIPVAIKELREATSPKANKEILDEAYVMASVDNPHVCRLLGICLTS
TVQLITQLMPFGCLLDYVREHKDNIGSQYLLNWCVQIAKGMNYLEDRRL
VHRDLAARNVLVKTPQHVKITDFGLAKLLGAEEKEYHAEGGKVPIKWMA
LESILHRIYTHQSDVWSYGVTVWELMTFGSKPYDGIPASEISSILEKGE
RLPQPPICTIDVYMIMVKCWMIDADSRPKFRELIIEFSKMARDPQRYLV
IQGDERMHLPSPTDSNFYRALMDEEDMDDVVDADEYLIPQQGFFSSPST
SRTPLLSSLSATSNNSTVACIDRNGLQSCPIKEDSFLQRYSSDPTGALT
EDSIDDTFLPVPEYINQSVPKRPAGSVQNPVYHNQPLNPAPSRDPHYQD
PHSTAVGNPEYLNTVQPTCVNSTFDSPAHWAQKGSHQISLDNPDYQQDF
FPKEAKPNGIFKGSTAENAEYLRVAPQSSEFIGA

The mature wild type EGFR may comprise (from the N to C-terminus) four extra-cellular domains, termed Domains I, II, Ill and IV; a Transmembrane Domain; a Juxtamembrane Domain; a Tyrosine Kinase Domain; and a Regulatory Region (see Ferguson, K. M. (2008) Annu Rev Biophys 37: 353-373). A skilled person would readily be able to identify the domains in an EGFR sequence using known sequence comparison tools.

For example, EGFR domains may be as follows in SEQ ID NO: 1 based on amino acid numbering following a convention wherein the N-terminal methionine of SEQ ID NO: 1 is assigned to be residue 1: signal peptide (amino acids 1-24); Domain I (amino acids 25-189); Domain II (amino acids 190-334); Domain III (amino acids 335-504); Domain IV (amino acids 505-644); Transmembrane Domain (amino acids 645-667); Juxtamembrane Domain (amino acids 668-709); Tyrosine Kinase Domain (amino acids 710-977); and Regulatory Region (amino acids 978-1210). A skilled person would readily be able to determine analogous domains in homologous proteins by performing a sequence alignment to SEQ ID NO: 1.

A functionally inert truncated version of epidermal growth factor receptor has been previously proposed as a candidate for selection, tracking and depletion of modified cells (Wang et al. (2011) Blood 118(5): 1255-1263). To generate the truncated EGFR in Wang et al., the wild-type receptor was rendered incapable of binding ligands and of signalling activity by removal of two extracellular domains (Domains I and II) and of its cytoplasmic tail (including the Juxtamembrane Domain and Tyrosine Kinase Domain).

The inventors have developed an improved truncated epidermal growth factor receptor, which exhibits increased stability and expression on the surface of cells even when its expression is driven by a weak promoter.

In one aspect the invention provides a polynucleotide comprising a nucleotide sequence encoding a truncated epidermal growth factor receptor (EGFRt), wherein the polynucleotide further comprises:
(a) a nucleotide sequence encoding a NGFR signal peptide; and/or
(b) a nucleotide sequence encoding a polypeptide comprising an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In another aspect the invention provides a polynucleotide comprising a nucleotide sequence encoding a truncated epidermal growth factor receptor (EGFRt), wherein the polynucleotide further comprises:
(a) a nucleotide sequence encoding a GMS SFR alpha signal peptide; and/or
(b) a nucleotide sequence encoding a polypeptide comprising an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In some embodiments the polynucleotide comprises a nucleotide sequence encoding a truncated epidermal growth factor receptor (EGFRt) and a nucleotide sequence encoding a NGFR signal peptide.

In some embodiments the polynucleotide comprises a nucleotide sequence encoding a truncated epidermal growth factor receptor (EGFRt) and a nucleotide sequence encoding a GMS SFR alpha signal peptide.

In some embodiments the polynucleotide comprises a nucleotide sequence encoding a truncated epidermal growth factor receptor (EGFRt) and a nucleotide sequence encoding a polypeptide comprising an amino acid sequence of KRWNRGIL (SEQ ID NO: 39) or a variant thereof having up to three amino acid substitutions, additions or deletions. In some embodiments the polynucleotide comprises a nucleotide sequence encoding a truncated epidermal growth factor receptor (EGFRt) and a nucleotide sequence encoding a polypeptide comprising an amino acid sequence of RRRHIVRK (SEQ ID NO: 40) or a variant thereof having up to three amino acid substitutions, additions or deletions.

The amino acid sequences of KRWNRGIL (SEQ ID NO: 39) or RRRHIVRK (SEQ ID NO: 40) may be referred to herein as a “cytoplasmic tail” or “cytoplasmic domain”. KRWNRGIL (SEQ ID NO: 39) may be referred to herein as a NGFR cytoplasmic tail. RRRHIVRK (SEQ ID NO: 40) may be referred to herein as a EGFR cytoplasmic tail.

In some embodiments the polynucleotide comprises a nucleotide sequence encoding a truncated epidermal growth factor receptor (EGFRt), wherein the polynucleotide further comprises: (a) a nucleotide sequence encoding a NGFR signal peptide; and (b) a nucleotide sequence encoding a polypeptide comprising an amino acid sequence of KRWNRGIL (SEQ ID NO: 39) or a variant thereof having up to three amino acid substitutions, additions or deletions. In some embodiments the polynucleotide comprises a nucleotide sequence encoding a truncated epidermal growth factor receptor (EGFRt), wherein the polynucleotide further comprises: (a) a nucleotide sequence encoding a NGFR signal peptide; and (b) a nucleotide sequence encoding a polypeptide comprising an amino acid sequence of RRRHIVRK (SEQ ID NO: 40) or a variant thereof having up to three amino acid substitutions, additions or deletions.

In some embodiments the polynucleotide comprises a nucleotide sequence encoding a truncated epidermal growth factor receptor (EGFRt), wherein the polynucleotide further comprises: (a) a nucleotide sequence encoding a GMS SFR alpha signal peptide; and (b) a nucleotide sequence encoding a polypeptide comprising an amino acid sequence of KRWNRGIL (SEQ ID NO: 39) or a variant thereof having up to three amino acid substitutions, additions or deletions. In some embodiments the polynucleotide comprises a nucleotide sequence encoding a truncated epidermal growth factor receptor (EGFRt), wherein the polynucleotide further comprises: (a) a nucleotide sequence encoding a GMS SFR alpha signal peptide; and (b) a nucleotide sequence encoding a polypeptide comprising an amino acid sequence of RRRHIVRK (SEQ ID NO: 40) or a variant thereof having up to three amino acid substitutions, additions or deletions.

Preferably the EGFRt of the invention is a truncated EGFR that comprises an EGFR extracellular epitope, which may be, for example, selectively bound by an anti-EGFR antibody, such as cetuximab. Preferably the EGFRt lacks signalling or trafficking activity.

In some embodiments the EGFRt comprises a EGFR Domain III and a EGFR Domain IV. In some embodiments the EGFRt further comprises a EGFR transmembrane domain or a NGFR transmembrane domain, preferably a EGFR transmembrane domain.

In some embodiments the EGFRt comprises a EGFR Domain III, a EGFR Domain IV and a EGFR transmembrane domain. In some embodiments the EGFRt consists of a EGFR Domain III, a EGFR Domain IV and a NGFR transmembrane domain.

In some embodiments the EGFRt does not comprise a EGFR Domain I. In some embodiments the EGFRt does not comprise a EGFR Domain II. In some embodiments the EGFRt does not comprise a EGFR Juxtamembrane Domain. In some embodiments the EGFRt does not comprise a EGFR Tyrosine Kinase Domain.

In some embodiments the EGFRt does not comprise a EGFR Domain I, a EGFR Domain II, a EGFR Juxtamembrane Domain or a EGFR Tyrosine Kinase Domain.

An example EGFRt amino acid sequence is:

(SEQ ID NO: 2)
RKVCNGIGIGEFKDSLSINATNIKHFKNCTSISGDLHILPVAFRGDSET
HTPPLDPQELDILKTVKEITGFLLIQAWPENRTDLHAFENLEIIRGRTK
QHGQFSLAVVSLNITSLGLRSLKEISDGDVIISGNKNLCYANTINWKKL
FGTSGQKTKIISNRGENSCKATGQVCHALCSPEGCWGPEPRDCVSCRNV
SRGRECVDKCNLLEGEPREFVENSECIQCHPECLPQAMNITCTGRGPDN
CIQCAHYIDGPHCVKTCPAGVMGENNTLVWKYADAGHVCHLCHPNCTYG
CTGPGLEGCPTNGPKIPSIATGMVGALLLLLVVALGIGLFM

An example nucleotide sequence encoding EGFRt is:

(SEQ ID NO: 3)
AGAAAAGTGTGCAACGGCATCGGCATCGGAGAGTTCAAGGACAGCCTGA
GCATCAACGCCACCAACATCAAGCACTTCAAGAACTGCACCAGCATCAG
CGGCGACCTGCACATTCTGCCTGTGGCCTTTAGAGGCGACAGCTTCACC
CACACACCTCCACTGGATCCCCAAGAGCTGGACATCCTGAAAACCGTGA
AAGAGATCACCGGATTTCTGTTGATCCAGGCTTGGCCCGAGAACCGGAC
AGATCTGCACGCCTTCGAGAACCTGGAAATCATCAGAGGCCGGACCAAG
CAGCACGGCCAGTTTTCTCTGGCTGTGGTGTCCCTGAACATCACCAGCC
TGGGCCTGAGAAGCCTGAAAGAAATCAGCGACGGCGACGTGATCATCTC
CGGCAACAAGAACCTGTGCTACGCCAACACCATCAACTGGAAGAAGCTG
TTCGGCACCAGCGGCCAGAAAACAAAGATCATCAGCAACCGGGGCGAGA
ACAGCTGCAAGGCTACAGGCCAAGTGTGCCACGCTCTGTGTAGCCCTGA
AGGCTGTTGGGGACCCGAGCCTAGAGATTGCGTGTCCTGCAGAAACGTG
TCCCGGGGCAGAGAATGCGTGGACAAGTGCAATCTGCTGGAAGGCGAGC
CCCGCGAGTTCGTGGAAAACAGCGAGTGCATCCAGTGTCACCCCGAGTG
TCTGCCCCAGGCCATGAACATTACCTGTACCGGCAGAGGCCCCGACAAC
TGTATTCAGTGCGCCCACTACATCGACGGCCCTCACTGCGTGAAAACAT
GTCCTGCTGGCGTGATGGGAGAGAACAACACCCTCGTGTGGAAGTATGC
CGACGCCGGACATGTGTGCCACCTGTGTCACCCTAATTGCACCTACGGC
TGTACAGGCCCTGGCCTGGAAGGCTGTCCAACAAACGGACCTAAGATCC
CCTCTATCGCCACCGGCATGGTTGGAGCCCTGCTGCTTCTGCTGGTGGT
GGCCCTTGGAATCGGCCTGTTTATG

In some embodiments the EGFRt comprises an amino acid sequence having at least 70%, 75%, 80%, 85%, 90%, 95%, 96%, 97%, 98% or 99% identity to SEQ ID NO: 2. In some embodiments the EGFRt consists of an amino acid sequence having at least 70%, 75%, 80%, 85%, 90%, 95%, 96%, 97%, 98% or 99% identity to SEQ ID NO: 2. Preferably the EGFRt comprises an epitope recognisable by an antibody, such as cetuximab. Preferably the EGFRt lacks signalling or trafficking activity.

In some embodiments the EGFRt comprises the amino acid sequence of SEQ ID NO: 2. In some embodiments the EGFRt consists of the amino acid sequence of SEQ ID NO: 2.

In some embodiments the EGFRt comprises an amino acid sequence having at least 70%, 75%, 80%, 85%, 90%, 95%, 96%, 97%, 98% or 99% identity to SEQ ID NO: 2 or a fragment thereof. In some embodiments the EGFRt consists of an amino acid sequence having at least 70%, 75%, 80%, 85%, 90%, 95%, 96%, 97%, 98% or 99% identity to SEQ ID NO: 2 or a fragment thereof. Preferably the EGFRt or fragment thereof comprises an epitope recognisable by an antibody, such as cetuximab. Preferably the EGFRt or fragment thereof lacks signalling or trafficking activity.

In some embodiments the EGFRt comprises the amino acid sequence of SEQ ID NO: 2 or a fragment thereof. In some embodiments the EGFRt consists of the amino acid sequence of SEQ ID NO: 2 or a fragment thereof.

In some embodiments the nucleotide sequence encoding the EGFRt is codon optimised. Preferably the nucleotide sequence encoding the EGFRt is codon optimised for expression in humans.

In some embodiments the nucleotide sequence encoding the EGFRt has at least 70%, 75%, 80%, 85%, 90%, 95%, 96%, 97%, 98% or 99% identity to SEQ ID NO: 3. Preferably the EGFRt comprises an epitope recognisable by an antibody, such as cetuximab. Preferably the EGFRt lacks signalling or trafficking activity.

In some embodiments the nucleotide sequence encoding the EGFRt is SEQ ID NO: 3.

In some embodiments the nucleotide sequence encoding the EGFRt has at least 70%, 75%, 80%, 85%, 90%, 95%, 96%, 97%, 98% or 99% identity to SEQ ID NO: 3 or a fragment thereof. Preferably the EGFRt or fragment thereof comprises an epitope recognisable by an antibody, such as cetuximab. Preferably the EGFRt or fragment thereof lacks signalling or trafficking activity.

In some embodiments the nucleotide sequence encoding the EGFRt is SEQ ID NO: 3 or a fragment thereof.

Signal Peptide

The polynucleotide preferably comprises a nucleotide sequence encoding a signal peptide, such as a NGFR or GMS SFR alpha signal peptide, preferably an NGFR signal peptide.

The terms “GMS SFR alpha signal peptide” or “NGFR signal peptide”, as used herein, may refer to signal peptides that are encoded by GMS SFR alpha or NGFR natural coding sequences, respectively. Such signal peptides direct expression of a protein to the cell surface. Signal peptides may be cleaved from the immature protein during processing.

In some embodiments the polynucleotide comprises a nucleotide sequence encoding a NGFR signal peptide.

An example NGFR signal peptide is:

(SEQ ID NO: 4)
MGAGATGRAMDGPRLLLLLLLGVSLGGA

An example nucleotide sequence encoding an NGFR signal peptide is:

(SEQ ID NO: 5)
ATGGGAGCTGGTGCTACCGGCAGAGCTATGGATGGACCTAGACTGCTGCTC
CTGCTGCTGCTCGGAGTTTCTCTTGGCGGAGCC

In some embodiments the NGFR signal peptide comprises an amino acid sequence having at least 70%, 75%, 80%, 85%, 90%, 95%, 96%, 97%, 98% or 99% identity to SEQ ID NO: 4. In some embodiments the NGFR signal peptide consists of an amino acid sequence having at least 70%, 75%, 80%, 85%, 90%, 95%, 96%, 97%, 98% or 99% identity to SEQ ID NO: 4. Preferably the NGFR signal peptide directs expression of the EGFRt to the cell surface.

In some embodiments the NGFR signal peptide comprises the amino acid sequence of SEQ ID NO: 4. In some embodiments the NGFR signal peptide consists of the amino acid sequence of SEQ ID NO: 4.

In some embodiments the nucleotide sequence encoding the NGFR signal peptide has at least 70%, 75%, 80%, 85%, 90%, 95%, 96%, 97%, 98% or 99% identity to SEQ ID NO: 5.

In some embodiments the nucleotide sequence encoding the NGFR signal peptide is SEQ ID NO: 5.

In some embodiments the NGFR signal peptide comprises an amino acid sequence having at least 70%, 75%, 80%, 85%, 90%, 95%, 96%, 97%, 98% or 99% identity to SEQ ID NO: 4 or a fragment thereof. In some embodiments the NGFR signal peptide consists of an amino acid sequence having at least 70%, 75%, 80%, 85%, 90%, 95%, 96%, 97%, 98% or 99% identity to SEQ ID NO: 4 or a fragment thereof. Preferably the NGFR signal peptide or fragment thereof directs expression of the EGFRt to the cell surface.

In some embodiments the NGFR signal peptide comprises the amino acid sequence of SEQ ID NO: 4 or a fragment thereof. In some embodiments the NGFR signal peptide consists of the amino acid sequence of SEQ ID NO: 4 or a fragment thereof.

In some embodiments the nucleotide sequence encoding the NGFR signal peptide has at least 70%, 75%, 80%, 85%, 90%, 95%, 96%, 97%, 98% or 99% identity to SEQ ID NO: 5 or a fragment thereof.

In some embodiments the nucleotide sequence encoding the NGFR signal peptide is SEQ ID NO: 5 or a fragment thereof.

In other embodiments the polynucleotide comprises a nucleotide sequence encoding a GMS SFR alpha signal peptide.

An example GMS SFR alpha signal peptide is (Wang et al. (2011) Blood 118: 1255-1263):

(SEQ ID NO: 6)
MLLLVTSLLLCELPHPAFLLIP

An example nucleotide sequence encoding an GMS SFR alpha signal peptide is:

(SEQ ID NO: 7)
ATGCTGCTGCTGGTCACCTCTCTGCTGCTGTGCGAACTGCCCCATCCTGC
CTTTCTGCTGATCCCC

In some embodiments the GMS SFR alpha signal peptide comprises an amino acid sequence having at least 70%, 75%, 80%, 85%, 90%, 95%, 96%, 97%, 98% or 99% identity to SEQ ID NO: 6. In some embodiments the GMS SFR alpha signal peptide consists of an amino acid sequence having at least 70%, 75%, 80%, 85%, 90%, 95%, 96%, 97%, 98% or 99% identity to SEQ ID NO: 6. Preferably the GMS SFR alpha signal peptide directs expression of the EGFRt to the cell surface.

In some embodiments the GMS SFR alpha signal peptide comprises the amino acid sequence of SEQ ID NO: 6. In some embodiments the GMS SFR alpha signal peptide consists of the amino acid sequence of SEQ ID NO: 6.

In some embodiments the nucleotide sequence encoding the GMS SFR alpha signal peptide has at least 70%, 75%, 80%, 85%, 90%, 95%, 96%, 97%, 98% or 99% identity to SEQ ID NO: 7.

In some embodiments the nucleotide sequence encoding the GMS SFR alpha signal peptide is SEQ ID NO: 7.

In some embodiments the GMS SFR alpha signal peptide comprises an amino acid sequence having at least 70%, 75%, 80%, 85%, 90%, 95%, 96%, 97%, 98% or 99% identity to SEQ ID NO: 6 or a fragment thereof. In some embodiments the GMS SFR alpha signal peptide consists of an amino acid sequence having at least 70%, 75%, 80%, 85%, 90%, 95%, 96%, 97%, 98% or 99% identity to SEQ ID NO: 6 or a fragment thereof. Preferably the GMS SFR alpha signal peptide or fragment thereof directs expression of the EGFRt to the cell surface.

In some embodiments the GMS SFR alpha signal peptide comprises the amino acid sequence of SEQ ID NO: 6 or a fragment thereof. In some embodiments the GMS SFR alpha signal peptide consists of the amino acid sequence of SEQ ID NO: 6 or a fragment thereof.

In some embodiments the nucleotide sequence encoding the GMS SFR alpha signal peptide has at least 70%, 75%, 80%, 85%, 90%, 95%, 96%, 97%, 98% or 99% identity to SEQ ID NO: 7 or a fragment thereof.

In some embodiments the nucleotide sequence encoding the GMS SFR alpha signal peptide is SEQ ID NO: 7 or a fragment thereof.

In preferred embodiments the signal peptide (e.g. the NGFR or GMS SFR alpha signal peptide, preferably the NGFR signal peptide) is operably linked to the EGFRt.

The term “operably linked”, as used herein, may mean that two components are linked together in a manner which enables both to carry out their function substantially unhindered. For example, the signal peptide may direct expression of the EGFRt to the cell surface.

For example, the signal peptide may be at the amino terminal end of the EGFRt. In some embodiments, the signal peptide is immediately to the amino terminus of the EGFRt.

Cytoplasmic Domain

The polynucleotide preferably comprises a nucleotide sequence encoding a cytoplasmic tail. Suitably the polynucleotide may comprise a nucleotide sequence encoding a polypeptide comprising an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

In some embodiments the variant of (i) or (ii) has three amino acid substitutions, additions or deletions. In some embodiments the variant of (i) or (ii) has two amino acid substitutions, additions or deletions. In some embodiments the variant of (i) or (ii) has one amino acid substitution, addition or deletion.

An example nucleotide sequence encoding (i) is:

(SEQ ID NO: 8)
AAGCGGTGGAACCGGGGCATCCTG

An example nucleotide sequence encoding (ii) is:

(SEQ ID NO: 9)
CGGCGGAGACACATCGTGCGGAAG

In some embodiments the nucleotide sequence encoding (i) or (ii) has at least 95%, 96%, 97%, 98% or 99% identity to SEQ ID NO: 8 or 9.

In some embodiments the nucleotide sequence encoding (i) or (ii) is SEQ ID NO: 8 or 9, respectively.

In some embodiments the nucleotide sequence encoding (i) or (ii) has at least 95%, 96%, 97%, 98% or 99% identity to SEQ ID NO: 8 or 9, or a fragment thereof.

In some embodiments the nucleotide sequence encoding (i) or (ii) is SEQ ID NO: 8 or 9, respectively, or a fragment thereof.

The cytoplasmic domain (e.g. (i), (ii) or variant thereof) may further comprise the amino acid sequence alanine-serine (AS) at the C-terminus. Thus, the invention further contemplates that SEQ ID NOs: 39 and 40 may be replaced by KRWNRGILAS (SEQ ID NO: 41) and RRRHIVRKAS (SEQ ID NO: 42), respectively. The “AS” sequence may be encoded by the nucleotide sequence GCTAGC. Thus, the invention further contemplates that SEQ ID NOs: 8 and 9 are replaced by AAGCGGTGGAACCGGGGCATCCTGGCTAGC (SEQ ID NO: 43) and CGGCGGAGACACATCGTGCGGAAGGCTAGC (SEQ ID NO: 44), respectively. The invention further contemplates that SEQ ID NOs: 8 and 9 are replaced by AAGCGGTGGAACCGGGGCATCCTGGCTAGC (SEQ ID NO: 43) and CGGCGGAGACACATCGTGCGGAAGGCTAGC (SEQ ID NO: 44), respectively, or fragments thereof.

Preferably the amino acid sequence of (i), (ii), or the variant thereof, is operably linked to the EGFRt. For example, the amino acid sequence of (i), (ii), or the variant thereof, may increase stability and expression of the EGFRt at the cell surface.

For example, the amino acid sequence of (i), (ii), or the variant thereof, may be at the carboxy terminal end of the EGFRt. In some embodiments the amino acid sequence of (i), (ii), or the variant thereof, is immediately to the carboxy terminus of the EGFRt.

In preferred embodiments the amino acid sequence of (i), (ii), or the variant thereof, may be at the carboxy terminal end of the EGFRt transmembrane domain. In preferred embodiments the amino acid sequence of (i), (ii), or the variant thereof, is immediately to the carboxy terminus of the EGFRt transmembrane domain. In other embodiments the amino acid sequence of (i), (ii), or the variant thereof, is joined to the carboxy terminal end of the EGFRt transmembrane domain by a linker, such as a linker peptide.

Transmembrane Domain

In some embodiments the polynucleotide comprises a nucleotide sequence encoding a EGFR transmembrane domain. In some embodiments the polynucleotide comprises a nucleotide sequence encoding a NGFR transmembrane domain.

An example EGFR transmembrane domain is:

(SEQ ID NO: 10)
IATGMVGALLLLLVVALGIGLEM

An example nucleotide sequence encoding an EGFR transmembrane domain is:

(SEQ ID NO: 11)
ATCGCCACCGGCATGGTTGGAGCCCTGCTGCTTCTGCTGGTGGTGGC
CCTTGGAATCGGCCTGTTTATG

In some embodiments the EGFR transmembrane domain comprises an amino acid sequence having at least 70%, 75%, 80%, 85%, 90%, 95%, 96%, 97%, 98% or 99% identity to SEQ ID NO: 10. In some embodiments the EGFR transmembrane domain consists of an amino acid sequence having at least 70%, 75%, 80%, 85%, 90%, 95%, 96%, 97%, 98% or 99% identity to SEQ ID NO: 10. Preferably the EGFR transmembrane domain anchors the EGFRt to the cell membrane.

In some embodiments the EGFR transmembrane domain comprises the amino acid sequence of SEQ ID NO: 10. In some embodiments the EGFR transmembrane domain consists of the amino acid sequence of SEQ ID NO: 10.

In some embodiments the nucleotide sequence encoding the EGFR transmembrane domain has at least 70%, 75%, 80%, 85%, 90%, 95%, 96%, 97%, 98% or 99% identity to SEQ ID NO: 11.

In some embodiments the nucleotide sequence encoding the EGFR transmembrane domain is SEQ ID NO: 11.

In some embodiments the EGFR transmembrane domain comprises an amino acid sequence having at least 70%, 75%, 80%, 85%, 90%, 95%, 96%, 97%, 98% or 99% identity to SEQ ID NO: 10 or a fragment thereof. In some embodiments the EGFR transmembrane domain consists of an amino acid sequence having at least 70%, 75%, 80%, 85%, 90%, 95%, 96%, 97%, 98% or 99% identity to SEQ ID NO: 10 or a fragment thereof. Preferably the EGFR transmembrane domain or fragment thereof anchors the EGFRt to the cell membrane.

In some embodiments the EGFR transmembrane domain comprises the amino acid sequence of SEQ ID NO: 10 or a fragment thereof. In some embodiments the EGFR transmembrane domain consists of the amino acid sequence of SEQ ID NO: 10 or a fragment thereof.

In some embodiments the nucleotide sequence encoding the EGFR transmembrane domain has at least 70%, 75%, 80%, 85%, 90%, 95%, 96%, 97%, 98% or 99% identity to SEQ ID NO: 11 or a fragment thereof.

In some embodiments the nucleotide sequence encoding the EGFR transmembrane domain is SEQ ID NO: 11 or a fragment thereof.

An example NGFR transmembrane domain is:

(SEQ ID NO: 12)
LIPVYCSILAAVVVGLVAYIAE

An example nucleotide sequence encoding an NGFR transmembrane domain is:

(SEQ ID NO: 13)
CTGATCCCCGTGTACTGTAGCATCCTGGCCGCCGTGGTTGTGGGA
CTCGTGGCCTATATCGCCTTC

In some embodiments the NGFR transmembrane domain comprises an amino acid sequence having at least 70%, 75%, 80%, 85%, 90%, 95%, 96%, 97%, 98% or 99% identity to SEQ ID NO: 12. In some embodiments the NGFR transmembrane domain consists of an amino acid sequence having at least 70%, 75%, 80%, 85%, 90%, 95%, 96%, 97%, 98% or 99% identity to SEQ ID NO: 12. Preferably the NGFR transmembrane domain anchors the EGFRt to the cell membrane.

In some embodiments the NGFR transmembrane domain comprises the amino acid sequence of SEQ ID NO: 12. In some embodiments the NGFR transmembrane domain consists of the amino acid sequence of SEQ ID NO: 12.

In some embodiments the nucleotide sequence encoding the NGFR transmembrane domain has at least 70%, 75%, 80%, 85%, 90%, 95%, 96%, 97%, 98% or 99% identity to SEQ ID NO: 13.

In some embodiments the nucleotide sequence encoding the NGFR transmembrane domain is SEQ ID NO: 13.

In some embodiments the NGFR transmembrane domain comprises an amino acid sequence having at least 70%, 75%, 80%, 85%, 90%, 95%, 96%, 97%, 98% or 99% identity to SEQ ID NO: 12 or a fragment thereof. In some embodiments the NGFR transmembrane domain consists of an amino acid sequence having at least 70%, 75%, 80%, 85%, 90%, 95%, 96%, 97%, 98% or 99% identity to SEQ ID NO: 12 or a fragment thereof. Preferably the NGFR transmembrane domain or fragment thereof anchors the EGFRt to the cell membrane.

In some embodiments the NGFR transmembrane domain comprises the amino acid sequence of SEQ ID NO: 12 or a fragment thereof. In some embodiments the NGFR transmembrane domain consists of the amino acid sequence of SEQ ID NO: 12 or a fragment thereof.

In some embodiments the nucleotide sequence encoding the NGFR transmembrane domain has at least 70%, 75%, 80%, 85%, 90%, 95%, 96%, 97%, 98% or 99% identity to SEQ ID NO: 13 or a fragment thereof.

In some embodiments the nucleotide sequence encoding the NGFR transmembrane domain is SEQ ID NO: 13 or a fragment thereof.

Recycling Signal

In some embodiments the polynucleotide further comprises a nucleotide sequence encoding a recycling signal. In other embodiments the polynucleotide does not comprise a nucleotide sequence encoding a recycling signal.

An example recycling signal sequence is:

(SEQ ID NO: 14)
YQPLSQIKRLLSDSFLEDNPVYAS

An example nucleotide sequence encoding a recycling signal is:

(SEQ ID NO: 15)
TATCAGCCTCTGAGCCAGATCAAGCGGCTGCTGAGCGACTCCTTCCT
GTTCGACAACCCCGTGTACGCTAGC

In some embodiments the recycling signal comprises an amino acid sequence having at least 70%, 75%, 80%, 85%, 90%, 95%, 96%, 97%, 98% or 99% identity to SEQ ID NO: 14. In some embodiments the recycling signal consists of an amino acid sequence having at least 70%, 75%, 80%, 85%, 90%, 95%, 96%, 97%, 98% or 99% identity to SEQ ID NO: 14. Preferably the recycling signal promotes recycling of the EGFRt to the cell membrane.

In some embodiments the recycling signal comprises the amino acid sequence of SEQ ID NO: 14. In some embodiments the recycling signal consists of the amino acid sequence of SEQ ID NO: 14.

In some embodiments the nucleotide sequence encoding the recycling signal has at least 70%, 75%, 80%, 85%, 90%, 95%, 96%, 97%, 98% or 99% identity to SEQ ID NO: 15.

In some embodiments the nucleotide sequence encoding the recycling signal is SEQ ID NO: 15.

In some embodiments the recycling signal comprises an amino acid sequence having at least 70%, 75%, 80%, 85%, 90%, 95%, 96%, 97%, 98% or 99% identity to SEQ ID NO: 14 or a fragment thereof. In some embodiments the recycling signal consists of an amino acid sequence having at least 70%, 75%, 80%, 85%, 90%, 95%, 96%, 97%, 98% or 99% identity to SEQ ID NO: 14 or a fragment thereof. Preferably the recycling signal or fragment thereof promotes recycling of the EGFRt to the cell membrane.

In some embodiments the recycling signal comprises the amino acid sequence of SEQ ID NO: 14 or a fragment thereof. In some embodiments the recycling signal consists of the amino acid sequence of SEQ ID NO: 14 or a fragment thereof.

In some embodiments the nucleotide sequence encoding the recycling signal has at least 70%, 75%, 80%, 85%, 90%, 95%, 96%, 97%, 98% or 99% identity to SEQ ID NO: 15 or a fragment thereof.

In some embodiments the nucleotide sequence encoding the recycling signal is SEQ ID NO: 15 or a fragment thereof.

Constructs

Example EGFRt constructs of the invention include:

| Description | Sequence | SEQ ID NO |
|---|---|---|
| Nucleotide sequence | ATGGGAGCTGGTGCTACCGGCAGAGCTATGGATGGACCTA | 16 |
| encoding modified | GACTGCTGCTCCTGCTGCTGCTCGGAGTTTCTCTTGGCGG | |
| EGFRt (with NGFR | AGCCAGAAAAGTGTGCAACGGCATCGGCATCGGAGAGTTC | |
| signal peptide) | AAGGACAGCCTGAGCATCAACGCCACCAACATCAAGCACT | |
| | TCAAGAACTGCACCAGCATCAGCGGCGACCTGCACATTCT | |
| | GCCTGTGGCCTTTAGAGGCGACAGCTTCACCCACACACCT | |
| | CCACTGGATCCCCAAGAGCTGGACATCCTGAAAACCGTGA | |
| | AAGAGATCACCGGATTTCTGTTGATCCAGGCTTGGCCCGA | |
| | GAACCGGACAGATCTGCACGCCTTCGAGAACCTGGAAATC | |
| | ATCAGAGGCCGGACCAAGCAGCACGGCCAGTTTTCTCTGG | |
| | CTGTGGTGTCCCTGAACATCACCAGCCTGGGCCTGAGAAG | |
| | CCTGAAAGAAATCAGCGACGGCGACGTGATCATCTCCGGC | |
| | AACAAGAACCTGTGCTACGCCAACACCATCAACTGGAAGA | |
| | AGCTGTTCGGCACCAGCGGCCAGAAAACAAAGATCATCAG | |
| | CAACCGGGGCGAGAACAGCTGCAAGGCTACAGGCCAAGTG | |
| | TGCCACGCTCTGTGTAGCCCTGAAGGCTGTTGGGGACCCG | |
| | AGCCTAGAGATTGCGTGTCCTGCAGAAACGTGTCCCGGGG | |
| | CAGAGAATGCGTGGACAAGTGCAATCTGCTGGAAGGCGAG | |
| | CCCCGCGAGTTCGTGGAAAACAGCGAGTGCATCCAGTGTC | |
| | ACCCCGAGTGTCTGCCCCAGGCCATGAACATTACCTGTAC | |
| | CGGCAGAGGCCCCGACAACTGTATTCAGTGCGCCCACTAC | |
| | ATCGACGGCCCTCACTGCGTGAAAACATGTCCTGCTGGCG | |
| | TGATGGGAGAGAACAACACCCTCGTGTGGAAGTATGCCGA | |
| | CGCCGGACATGTGTGCCACCTGTGTCACCCTAATTGCACC | |
| | TACGGCTGTACAGGCCCTGGCCTGGAAGGCTGTCCAACAA | |
| | ACGGACCTAAGATCCCCTCTATCGCCACCGGCATGGTTGG | |
| | AGCCCTGCTGCTTCTGCTGGTGGTGGCCCTTGGAATCGGC | |
| | CTGTTTATG | |
| Modified EGFRt (with | MGAGATGRAMDGPRLLLLLLLGVSLGGARKVCNGIGIGEF | 17 |
| NGFR signal peptide) | KDSLSINATNIKHFKNCTSISGDLHILPVAFRGDSFTHTP | |
| | PLDPQELDILKTVKEITGFLLIQAWPENRTDLHAFENLEI | |
| | IRGRTKQHGQFSLAVVSLNITSLGLRSLKEISDGDVIISG | |
| | NKNLCYANTINWKKLFGTSGQKTKIISNRGENSCKATGQV | |
| | CHALCSPEGCWGPEPRDCVSCRNVSRGRECVDKCNLLEGE | |
| | PREFVENSECIQCHPECLPQAMNITCTGRGPDNCIQCAHY | |
| | IDGPHCVKTCPAGVMGENNTLVWKYADAGHVCHLCHPNCT | |
| | YGCTGPGLEGCPTNGPKIPSIATGMVGALLLLLVVALGIG | |
| | LFM | |
| Nucleotide sequence | AGAAAAGTGTGCAACGGCATCGGCATCGGAGAGTTCAAGG | 18 |
| encoding modified | ACAGCCTGAGCATCAACGCCACCAACATCAAGCACTTCAA | |
| EGFRt (with NGFR | GAACTGCACCAGCATCAGCGGCGACCTGCACATTCTGCCT | |
| cytoplasmic domain) | GTGGCCTTTAGAGGCGACAGCTTCACCCACACACCTCCAC | |
| | TGGATCCCCAAGAGCTGGACATCCTGAAAACCGTGAAAGA | |
| | GATCACCGGATTTCTGTTGATCCAGGCTTGGCCCGAGAAC | |
| | CGGACAGATCTGCACGCCTTCGAGAACCTGGAAATCATCA | |
| | GAGGCCGGACCAAGCAGCACGGCCAGTTTTCTCTGGCTGT | |
| | GGTGTCCCTGAACATCACCAGCCTGGGCCTGAGAAGCCTG | |
| | AAAGAAATCAGCGACGGCGACGTGATCATCTCCGGCAACA | |
| | AGAACCTGTGCTACGCCAACACCATCAACTGGAAGAAGCT | |
| | GTTCGGCACCAGCGGCCAGAAAACAAAGATCATCAGCAAC | |
| | CGGGGCGAGAACAGCTGCAAGGCTACAGGCCAAGTGTGCC | |
| | ACGCTCTGTGTAGCCCTGAAGGCTGTTGGGGACCCGAGCC | |
| | TAGAGATTGCGTGTCCTGCAGAAACGTGTCCCGGGGCAGA | |
| | GAATGCGTGGACAAGTGCAATCTGCTGGAAGGCGAGCCCC | |
| | GCGAGTTCGTGGAAAACAGCGAGTGCATCCAGTGTCACCC | |
| | CGAGTGTCTGCCCCAGGCCATGAACATTACCTGTACCGGC | |
| | AGAGGCCCCGACAACTGTATTCAGTGCGCCCACTACATCG | |
| | ACGGCCCTCACTGCGTGAAAACATGTCCTGCTGGCGTGAT | |
| | GGGAGAGAACAACACCCTCGTGTGGAAGTATGCCGACGCC | |
| | GGACATGTGTGCCACCTGTGTCACCCTAATTGCACCTACG | |
| | GCTGTACAGGCCCTGGCCTGGAAGGCTGTCCAACAAACGG | |
| | ACCTAAGATCCCCTCTATCGCCACCGGCATGGTTGGAGCC | |
| | CTGCTGCTTCTGCTGGTGGTGGCCCTTGGAATCGGCCTGT | |
| | TTATGAAGCGGTGGAACCGGGGCATCCTGGCTAGCTGA | |
| Modified EGFRt (with | RKVCNGIGIGEFKDSLSINATNIKHFKNCTSISGDLHILP | 19 |
| NGFR cytoplasmic | VAFRGDSFTHTPPLDPQELDILKTVKEITGFLLIQAWPEN | |
| domain) | RTDLHAFENLEIIRGRTKQHGQFSLAVVSLNITSLGLRSL | |
| | KEISDGDVIISGNKNLCYANTINWKKLFGTSGQKTKIISN | |
| | RGENSCKATGQVCHALCSPEGCWGPEPRDCVSCRNVSRGR | |
| | ECVDKCNLLEGEPREFVENSECIQCHPECLPQAMNITCTG | |
| | RGPDNCIQCAHYIDGPHCVKTCPAGVMGENNTLVWKYADA | |
| | GHVCHLCHPNCTYGCTGPGLEGCPTNGPKIPSIATGMVGA | |
| | LLLLLVVALGIGLFMKRWNRGILAS | |
| Nucleotide sequence | AGAAAAGTGTGCAACGGCATCGGCATCGGAGAGTTCAAGG | 20 |
| encoding modified | ACAGCCTGAGCATCAACGCCACCAACATCAAGCACTTCAA | |
| EGFRt (with EGFR | GAACTGCACCAGCATCAGCGGCGACCTGCACATTCTGCCT | |
| cytoplasmic domain) | GTGGCCTTTAGAGGCGACAGCTTCACCCACACACCTCCAC | |
| | TGGATCCCCAAGAGCTGGACATCCTGAAAACCGTGAAAGA | |
| | GATCACCGGATTTCTGTTGATCCAGGCTTGGCCCGAGAAC | |
| | CGGACAGATCTGCACGCCTTCGAGAACCTGGAAATCATCA | |
| | GAGGCCGGACCAAGCAGCACGGCCAGTTTTCTCTGGCTGT | |
| | GGTGTCCCTGAACATCACCAGCCTGGGCCTGAGAAGCCTG | |
| | AAAGAAATCAGCGACGGCGACGTGATCATCTCCGGCAACA | |
| | AGAACCTGTGCTACGCCAACACCATCAACTGGAAGAAGCT | |
| | GTTCGGCACCAGCGGCCAGAAAACAAAGATCATCAGCAAC | |
| | CGGGGCGAGAACAGCTGCAAGGCTACAGGCCAAGTGTGCC | |
| | ACGCTCTGTGTAGCCCTGAAGGCTGTTGGGGACCCGAGCC | |
| | TAGAGATTGCGTGTCCTGCAGAAACGTGTCCCGGGGCAGA | |
| | GAATGCGTGGACAAGTGCAATCTGCTGGAAGGCGAGCCCC | |
| | GCGAGTTCGTGGAAAACAGCGAGTGCATCCAGTGTCACCC | |
| | CGAGTGTCTGCCCCAGGCCATGAACATTACCTGTACCGGC | |
| | AGAGGCCCCGACAACTGTATTCAGTGCGCCCACTACATCG | |
| | ACGGCCCTCACTGCGTGAAAACATGTCCTGCTGGCGTGAT | |
| | GGGAGAGAACAACACCCTCGTGTGGAAGTATGCCGACGCC | |
| | GGACATGTGTGCCACCTGTGTCACCCTAATTGCACCTACG | |
| | GCTGTACAGGCCCTGGCCTGGAAGGCTGTCCAACAAACGG | |
| | ACCTAAGATCCCCTCTATCGCCACCGGCATGGTTGGAGCC | |
| | CTGCTGCTTCTGCTGGTGGTGGCCCTTGGAATCGGCCTGT | |
| | TTATGCGGCGGAGACACATCGTGCGGAAGGCTAGCTGA | |
| Modified EGFRt (with | RKVCNGIGIGEFKDSLSINATNIKHFKNCTSISGDLHILP | 21 |
| EGFR cytoplasmic | VAFRGDSFTHTPPLDPQELDILKTVKEITGFLLIQAWPEN | |
| domain) | RTDLHAFENLEIIRGRTKQHGQFSLAVVSLNITSLGLRSL | |
| | KEISDGDVIISGNKNLCYANTINVKKLFGTSGQKTKIISN | |
| | RGENSCKATGQVCHALCSPEGCWGPEPRDCVSCRNVSRGR | |
| | ECVDKCNLLEGEPREFVENSECIQCHPECLPQAMNITCTG | |
| | RGPDNCIQCAHYIDGPHCVKTCPAGVMGENNTLVWKYADA | |
| | GHVCHLCHPNCTYGCTGPGLEGCPTNGPKIPSIATGMVGA | |
| | LLLLLVVALGIGLFMRRRHIVRKAS | |
| Nucleotide sequence | ATGGGAGCTGGTGCTACCGGCAGAGCTATGGATGGACCTA | 22 |
| encoding modified | GACTGCTGCTCCTGCTGCTGCTCGGAGTTTCTCTTGGCGG | |
| EGFRt (with NGFR | AGCCAGAAAAGTGTGCAACGGCATCGGCATCGGAGAGTTC | |
| signal peptide and | AAGGACAGCCTGAGCATCAACGCCACCAACATCAAGCACT | |
| NGFR cytoplasmic | TCAAGAACTGCACCAGCATCAGCGGCGACCTGCACATTCT | |
| domain) | GCCTGTGGCCTTTAGAGGCGACAGCTTCACCCACACACCT | |
| | CCACTGGATCCCCAAGAGCTGGACATCCTGAAAACCGTGA | |
| | AAGAGATCACCGGATTTCTGTTGATCCAGGCTTGGCCCGA | |
| | GAACCGGACAGATCTGCACGCCTTCGAGAACCTGGAAATC | |
| | ATCAGAGGCCGGACCAAGCAGCACGGCCAGTTTTCTCTGG | |
| | CTGTGGTGTCCCTGAACATCACCAGCCTGGGCCTGAGAAG | |
| | CCTGAAAGAAATCAGCGACGGCGACGTGATCATCTCCGGC | |
| | AACAAGAACCTGTGCTACGCCAACACCATCAACTGGAAGA | |
| | AGCTGTTCGGCACCAGCGGCCAGAAAACAAAGATCATCAG | |
| | CAACCGGGGCGAGAACAGCTGCAAGGCTACAGGCCAAGTG | |
| | TGCCACGCTCTGTGTAGCCCTGAAGGCTGTTGGGGACCCG | |
| | AGCCTAGAGATTGCGTGTCCTGCAGAAACGTGTCCCGGGG | |
| | CAGAGAATGCGTGGACAAGTGCAATCTGCTGGAAGGCGAG | |
| | CCCCGCGAGTTCGTGGAAAACAGCGAGTGCATCCAGTGTC | |
| | ACCCCGAGTGTCTGCCCCAGGCCATGAACATTACCTGTAC | |
| | CGGCAGAGGCCCCGACAACTGTATTCAGTGCGCCCACTAC | |
| | ATCGACGGCCCTCACTGCGTGAAAACATGTCCTGCTGGCG | |
| | TGATGGGAGAGAACAACACCCTCGTGTGGAAGTATGCCGA | |
| | CGCCGGACATGTGTGCCACCTGTGTCACCCTAATTGCACC | |
| | TACGGCTGTACAGGCCCTGGCCTGGAAGGCTGTCCAACAA | |
| | ACGGACCTAAGATCCCCTCTATCGCCACCGGCATGGTTGG | |
| | AGCCCTGCTGCTTCTGCTGGTGGTGGCCCTTGGAATCGGC | |
| | CTGTTTATGAAGCGGTGGAACCGGGGCATCCTGGCTAGCT | |
| | GA | |
| Modified EGFRt (with | MGAGATGRAMDGPRLLLLLLLGVSLGGARKVCNGIGIGEF | 23 |
| NGFR signal peptide | KDSLSINATNIKHFKNCTSISGDLHILPVAFRGDSFTHTP | |
| and NGFR | PLDPQELDILKTVKEITGFLLIQAWPENRTDLHAFENLEI | |
| cytoplasmic domain) | IRGRTKQHGQFSLAVVSLNITSLGLRSLKEISDGDVIISG | |
| | NKNLCYANTINWKKLFGTSGQKTKIISNRGENSCKATGQV | |
| | CHALCSPEGCWGPEPRDCVSCRNVSRGRECVDKCNLLEGE | |
| | PREFVENSECIQCHPECLPQAMNITCTGRGPDNCIQCAHY | |
| | IDGPHCVKTCPAGVMGENNTLVWKYADAGHVCHLCHPNCT | |
| | YGCTGPGLEGCPTNGPKIPSIATGMVGALLLLLVVALGIG | |
| | LFMKRWNRGILAS | |
| Nucleotide sequence | ATGGGAGCTGGTGCTACCGGCAGAGCTATGGATGGACCTA | 24 |
| encoding modified | GACTGCTGCTCCTGCTGCTGCTCGGAGTTTCTCTTGGCGG | |
| EGFRt (with NGFR | AGCCAGAAAAGTGTGCAACGGCATCGGCATCGGAGAGTTC | |
| signal peptide and | AAGGACAGCCTGAGCATCAACGCCACCAACATCAAGCACT | |
| EGFR cytoplasmic | TCAAGAACTGCACCAGCATCAGCGGCGACCTGCACATTCT | |
| domain) | GCCTGTGGCCTTTAGAGGCGACAGCTTCACCCACACACCT | |
| | CCACTGGATCCCCAAGAGCTGGACATCCTGAAAACCGTGA | |
| | AAGAGATCACCGGATTTCTGTTGATCCAGGCTTGGCCCGA | |
| | GAACCGGACAGATCTGCACGCCTTCGAGAACCTGGAAATC | |
| | ATCAGAGGCCGGACCAAGCAGCACGGCCAGTTTTCTCTGG | |
| | CTGTGGTGTCCCTGAACATCACCAGCCTGGGCCTGAGAAG | |
| | CCTGAAAGAAATCAGCGACGGCGACGTGATCATCTCCGGC | |
| | AACAAGAACCTGTGCTACGCCAACACCATCAACTGGAAGA | |
| | AGCTGTTCGGCACCAGCGGCCAGAAAACAAAGATCATCAG | |
| | CAACCGGGGCGAGAACAGCTGCAAGGCTACAGGCCAAGTG | |
| | TGCCACGCTCTGTGTAGCCCTGAAGGCTGTTGGGGACCCG | |
| | AGCCTAGAGATTGCGTGTCCTGCAGAAACGTGTCCCGGGG | |
| | CAGAGAATGCGTGGACAAGTGCAATCTGCTGGAAGGCGAG | |
| | CCCCGCGAGTTCGTGGAAAACAGCGAGTGCATCCAGTGTC | |
| | ACCCCGAGTGTCTGCCCCAGGCCATGAACATTACCTGTAC | |
| | CGGCAGAGGCCCCGACAACTGTATTCAGTGCGCCCACTAC | |
| | ATCGACGGCCCTCACTGCGTGAAAACATGTCCTGCTGGCG | |
| | TGATGGGAGAGAACAACACCCTCGTGTGGAAGTATGCCGA | |
| | CGCCGGACATGTGTGCCACCTGTGTCACCCTAATTGCACC | |
| | TACGGCTGTACAGGCCCTGGCCTGGAAGGCTGTCCAACAA | |
| | ACGGACCTAAGATCCCCTCTATCGCCACCGGCATGGTTGG | |
| | AGCCCTGCTGCTTCTGCTGGTGGTGGCCCTTGGAATCGGC | |
| | CTGTTTATGCGGCGGAGACACATCGTGCGGAAGGCTAGCT | |
| | GA | |
| Modified EGFRt (with | MGAGATGRAMDGPRLLLLLLLGVSLGGARKVCNGIGIGEF | 25 |
| NGFR signal peptide | KDSLSINATNIKHFKNCTSISGDLHILPVAFRGDSFTHTP | |
| and EGFR cytoplasmic | PLDPQELDILKTVKEITGFLLIQAWPENRTDLHAFENLEI | |
| domain) | IRGRTKQHGQFSLAVVSLNITSLGLRSLKEISDGDVIISG | |
| | NKNLCYANTINWKKLFGTSGQKTKIISNRGENSCKATGQV | |
| | CHALCSPEGCWGPEPRDCVSCRNVSRGRECVDKCNLLEGE | |
| | PREFVENSECIQCHPECLPQAMNITCTGRGPDNCIQCAHY | |
| | IDGPHCVKTCPAGVMGENNTLVWKYADAGHVCHLCHPNCT | |
| | YGCTGPGLEGCPTNGPKIPSIATGMVGALLLLLVVALGIG | |
| | LFMRRRHIVRKAS | |

In some embodiments the polynucleotide encodes an amino acid sequence having at least 70%, 75%, 80%, 85%, 90%, 95%, 96%, 97%, 98% or 99% identity to SEQ ID NO: 17, 19, 21, 23 or 25.

In some embodiments the polynucleotide encodes the amino acid sequence of SEQ ID NO: 17, 19, 21, 23 or 25.

In some embodiments the polynucleotide comprises a nucleotide sequence having at least 70%, 75%, 80%, 85%, 90%, 95%, 96%, 97%, 98% or 99% identity to SEQ ID NO: 16, 18, 20, 22 or 24.

In some embodiments the polynucleotide comprises a nucleotide sequence of SEQ ID NO: 16, 18, 20, 22 or 24.

In some embodiments the polynucleotide consists of a nucleotide sequence having at least 70%, 75%, 80%, 85%, 90%, 95%, 96%, 97%, 98% or 99% identity to SEQ ID NO: 16, 18, 20, 22 or 24.

In some embodiments the polynucleotide consists of a nucleotide sequence of SEQ ID NO: 16, 18, 20, 22 or 24.

In some embodiments the polynucleotide encodes an amino acid sequence having at least 70%, 75%, 80%, 85%, 90%, 95%, 96%, 97%, 98% or 99% identity to SEQ ID NO: 17, 19, 21, 23 or 25, or a fragment thereof.

In some embodiments the polynucleotide encodes the amino acid sequence of SEQ ID NO: 17, 19, 21, 23 or 25, or a fragment thereof.

In some embodiments the polynucleotide comprises a nucleotide sequence having at least 70%, 75%, 80%, 85%, 90%, 95%, 96%, 97%, 98% or 99% identity to SEQ ID NO: 16, 18, 20, 22 or 24, or a fragment thereof.

In some embodiments the polynucleotide comprises a nucleotide sequence of SEQ ID NO: 16, 18, 20, 22 or 24, or a fragment thereof.

In some embodiments the polynucleotide consists of a nucleotide sequence having at least 70%, 75%, 80%, 85%, 90%, 95%, 96%, 97%, 98% or 99% identity to SEQ ID NO: 16, 18, 20, 22 or 24, or a fragment thereof.

In some embodiments the polynucleotide consists of a nucleotide sequence of SEQ ID NO: 16, 18, 20, 22 or 24, or a fragment thereof.

Further example EGFRt constructs include:

| Description | Sequence | SEQ ID NO |
|---|---|---|
| Comparative | ATGCTGCTGCTGGTCACCTCTCTGCTGCTGTGCGAACTGC | 26 |
| nucleotide sequence | CCCATCCTGCCTTTCTGCTGATCCCCAGAAAAGTGTGCAA | |
| encoding an EGFRt | CGGCATCGGCATCGGAGAGTTCAAGGACAGCCTGAGCATC | |
| | AACGCCACCAACATCAAGCACTTCAAGAACTGCACCAGCA | |
| | TCAGCGGCGACCTGCACATTCTGCCTGTGGCCTTTAGAGG | |
| | CGACAGCTTCACCCACACACCTCCACTGGATCCCCAAGAG | |
| | CTGGACATCCTGAAAACCGTGAAAGAGATCACCGGATTTC | |
| | TGTTGATCCAGGCTTGGCCCGAGAACCGGACAGATCTGCA | |
| | CGCCTTCGAGAACCTGGAAATCATCAGAGGCCGGACCAAG | |
| | CAGCACGGCCAGTTTTCTCTGGCTGTGGTGTCCCTGAACA | |
| | TCACCAGCCTGGGCCTGAGAAGCCTGAAAGAAATCAGCGA | |
| | CGGCGACGTGATCATCTCCGGCAACAAGAACCTGTGCTAC | |
| | GCCAACACCATCAACTGGAAGAAGCTGTTCGGCACCAGCG | |
| | GCCAGAAAACAAAGATCATCAGCAACCGGGGCGAGAACAG | |
| | CTGCAAGGCTACAGGCCAAGTGTGCCACGCTCTGTGTAGC | |
| | CCTGAAGGCTGTTGGGGACCCGAGCCTAGAGATTGCGTGT | |
| | CCTGCAGAAACGTGTCCCGGGGCAGAGAATGCGTGGACAA | |
| | GTGCAATCTGCTGGAAGGCGAGCCCCGCGAGTTCGTGGAA | |
| | AACAGCGAGTGCATCCAGTGTCACCCCGAGTGTCTGCCCC | |
| | AGGCCATGAACATTACCTGTACCGGCAGAGGCCCCGACAA | |
| | CTGTATTCAGTGCGCCCACTACATCGACGGCCCTCACTGC | |
| | GTGAAAACATGTCCTGCTGGCGTGATGGGAGAGAACAACA | |
| | CCCTCGTGTGGAAGTATGCCGACGCCGGACATGTGTGCCA | |
| | CCTGTGTCACCCTAATTGCACCTACGGCTGTACAGGCCCT | |
| | GGCCTGGAAGGCTGTCCAACAAACGGACCTAAGATCCCCT | |
| | CTATCGCCACCGGCATGGTTGGAGCCCTGCTGCTTCTGCT | |
| | GGTGGTGGCCCTTGGAATCGGCCTGTTTATGTAG | |
| Comparative EGFRt | MLLLVTSLLLCELPHPAFLLIPRKVCNGIGIGEFKDSLSI | 27 |
| sequence | NATNIKHFKNCTSISGDLHILPVAFRGDSFTHTPPLDPQE | |
| | LDILKTVKEITGFLLIQAWPENRTDLHAFENLEIIRGRTK | |
| | QHGQFSLAVVSLNITSLGLRSLKEISDGDVIISGNKNLCY | |
| | ANTINWKKLFGTSGQKTKIISNRGENSCKATGQVCHALCS | |
| | PEGCWGPEPRDCVSCRNVSRGRECVDKCNLLEGEPREFVE | |
| | NSECIQCHPECLPQAMNITCTGRGPDNCIQCAHYIDGPHC | |
| | VKTCPAGVMGENNTLVWKYADAGHVCHLCHPNCTYGCTGP | |
| | GLEGCPTNGPKIPSIATGMVGALLLLLVVALGIGLFM | |
| Nucleotide sequence | ATGCTGCTGCTGGTCACCTCTCTGCTGCTGTGCGAACTGC | 28 |
| encoding modified | CCCATCCTGCCTTTCTGCTGATCCCCAGAAAAGTGTGCAA | |
| EGFRt (with GMS | CGGCATCGGCATCGGAGAGTTCAAGGACAGCCTGAGCATC | |
| SFR alpha signal | AACGCCACCAACATCAAGCACTTCAAGAACTGCACCAGCA | |
| peptide; and NGFR | TCAGCGGCGACCTGCACATTCTGCCTGTGGCCTTTAGAGG | |
| transmembrane and | CGACAGCTTCACCCACACACCTCCACTGGATCCCCAAGAG | |
| cytoplasmic domain) | CTGGACATCCTGAAAACCGTGAAAGAGATCACCGGATTTC | |
| | TGTTGATCCAGGCTTGGCCCGAGAACCGGACAGATCTGCA | |
| | CGCCTTCGAGAACCTGGAAATCATCAGAGGCCGGACCAAG | |
| | CAGCACGGCCAGTTTTCTCTGGCTGTGGTGTCCCTGAACA | |
| | TCACCAGCCTGGGCCTGAGAAGCCTGAAAGAAATCAGCGA | |
| | CGGCGACGTGATCATCTCCGGCAACAAGAACCTGTGCTAC | |
| | GCCAACACCATCAACTGGAAGAAGCTGTTCGGCACCAGCG | |
| | GCCAGAAAACAAAGATCATCAGCAACCGGGGCGAGAACAG | |
| | CTGCAAGGCTACAGGCCAAGTGTGCCACGCTCTGTGTAGC | |
| | CCTGAAGGCTGTTGGGGACCCGAGCCTAGAGATTGCGTGT | |
| | CCTGCAGAAACGTGTCCCGGGGCAGAGAATGCGTGGACAA | |
| | GTGCAATCTGCTGGAAGGCGAGCCCCGCGAGTTCGTGGAA | |
| | AACAGCGAGTGCATCCAGTGTCACCCCGAGTGTCTGCCCC | |
| | AGGCCATGAACATTACCTGTACCGGCAGAGGCCCCGACAA | |
| | CTGTATTCAGTGCGCCCACTACATCGACGGCCCTCACTGC | |
| | GTGAAAACATGTCCTGCTGGCGTGATGGGAGAGAACAACA | |
| | CCCTCGTGTGGAAGTATGCCGACGCCGGACATGTGTGCCA | |
| | CCTGTGTCACCCTAATTGCACCTACGGCTGTACAGGCCCT | |
| | GGCCTGGAAGGCTGTCCAACAAACGGACCTAAGATCCCCT | |
| | CTGGCGGCGGAGGATCTGGCGGAGGTGGAAGCGGAGGCGG | |
| | TGGATCTCTGATCCCCGTGTACTGTAGCATCCTGGCCGCC | |
| | GTGGTTGTGGGACTCGTGGCCTATATCGCCTTCAAGCGGT | |
| | GGAACCGGGGCATCCTGGCTAGCTGA | |
| Modified EGFRt (with | MLLLVTSLLLCELPHPAFLLIPRKVCNGIGIGEFKDSLSI | 29 |
| GMS SFR alpha signal | NATNIKHFKNCTSISGDLHILPVAFRGDSFTHTPPLDPQE | |
| peptide; and NGFR | LDILKTVKEITGFLLIQAWPENRTDLHAFENLEIIRGRTK | |
| transmembrane and | QHGQFSLAVVSLNITSLGLRSLKEISDGDVIISGNKNLCY | |
| cytoplasmic domain) | ANTINWKKLFGTSGQKTKIISNRGENSCKATGQVCHALCS | |
| | PEGCWGPEPRDCVSCRNVSRGRECVDKCNLLEGEPREFVE | |
| | NSECIQCHPECLPQAMNITCTGRGPDNCIQCAHYIDGPHC | |
| | VKTCPAGVMGENNTLVWKYADAGHVCHLCHPNCTYGCTGP | |
| | GLEGCPTNGPKIPSGGGGSGGGGSGGGGSLIPVYCSILAA | |
| | VVVGLVAYIAFKRWNRGILAS | |
| Nucleotide sequence | ATGGGAGCTGGTGCTACCGGCAGAGCTATGGATGGACCTA | 30 |
| encoding modified | GACTGCTGCTCCTGCTGCTGCTCGGAGTTTCTCTTGGCGG | |
| EGFRt (with NGFR | AGCCAGAAAAGTGTGCAACGGCATCGGCATCGGAGAGTTC | |
| signal peptide, NGFR | AAGGACAGCCTGAGCATCAACGCCACCAACATCAAGCACT | |
| cytoplasmic domain | TCAAGAACTGCACCAGCATCAGCGGCGACCTGCACATTCT | |
| and recycling signal) | GCCTGTGGCCTTTAGAGGCGACAGCTTCACCCACACACCT | |
| | CCACTGGATCCCCAAGAGCTGGACATCCTGAAAACCGTGA | |
| | AAGAGATCACCGGATTTCTGTTGATCCAGGCTTGGCCCGA | |
| | GAACCGGACAGATCTGCACGCCTTCGAGAACCTGGAAATC | |
| | ATCAGAGGCCGGACCAAGCAGCACGGCCAGTTTTCTCTGG | |
| | CTGTGGTGTCCCTGAACATCACCAGCCTGGGCCTGAGAAG | |
| | CCTGAAAGAAATCAGCGACGGCGACGTGATCATCTCCGGC | |
| | AACAAGAACCTGTGCTACGCCAACACCATCAACTGGAAGA | |
| | AGCTGTTCGGCACCAGCGGCCAGAAAACAAAGATCATCAG | |
| | CAACCGGGGCGAGAACAGCTGCAAGGCTACAGGCCAAGTG | |
| | TGCCACGCTCTGTGTAGCCCTGAAGGCTGTTGGGGACCCG | |
| | AGCCTAGAGATTGCGTGTCCTGCAGAAACGTGTCCCGGGG | |
| | CAGAGAATGCGTGGACAAGTGCAATCTGCTGGAAGGCGAG | |
| | CCCCGCGAGTTCGTGGAAAACAGCGAGTGCATCCAGTGTC | |
| | ACCCCGAGTGTCTGCCCCAGGCCATGAACATTACCTGTAC | |
| | CGGCAGAGGCCCCGACAACTGTATTCAGTGCGCCCACTAC | |
| | ATCGACGGCCCTCACTGCGTGAAAACATGTCCTGCTGGCG | |
| | TGATGGGAGAGAACAACACCCTCGTGTGGAAGTATGCCGA | |
| | CGCCGGACATGTGTGCCACCTGTGTCACCCTAATTGCACC | |
| | TACGGCTGTACAGGCCCTGGCCTGGAAGGCTGTCCAACAA | |
| | ACGGACCTAAGATCCCCTCTATCGCCACCGGCATGGTTGG | |
| | AGCCCTGCTGCTTCTGCTGGTGGTGGCCCTTGGAATCGGC | |
| | CTGTTTATGAAGCGGTGGAACCGGGGCATCCTGGCTAGCT | |
| | ATCAGCCTCTGAGCCAGATCAAGCGGCTGCTGAGCGACTC | |
| | CTTCCTGTTCGACAACCCCGTGTACGCTAGCTGA | |
| Modified EGFRt (with | MGAGATGRAMDGPRLLLLLLLGVSLGGARKVCNGIGIGEF | 31 |
| NGFR signal peptide, | KDSLSINATNIKHFKNCTSISGDLHILPVAFRGDSFTHTP | |
| NGFR cytoplasmic | PLDPQELDILKTVKEITGFLLIQAWPENRTDLHAFENLEI | |
| domain and recycling | IRGRTKQHGQFSLAVVSLNITSLGLRSLKEISDGDVIISG | |
| signal) | NKNLCYANTINWKKLFGTSGQKTKIISNRGENSCKATGQV | |
| | CHALCSPEGCWGPEPRDCVSCRNVSRGRECVDKCNLLEGE | |
| | PREFVENSECIQCHPECLPQAMNITCTGRGPDNCIQCAHY | |
| | IDGPHCVKTCPAGVMGENNTLVWKYADAGHVCHLCHPNCT | |
| | YGCTGPGLEGCPTNGPKIPSIATGMVGALLLLLVVALGIG | |
| | LFMKRWNRGILASYQPLSQIKRLLSDSFLFDNPVYAS | |
| Nucleotide sequence | ATGGGAGCTGGTGCTACCGGCAGAGCTATGGATGGACCTA | 32 |
| encoding modified | GACTGCTGCTCCTGCTGCTGCTCGGAGTTTCTCTTGGCGG | |
| EGFRt (with NGFR | AGCCAGAAAAGTGTGCAACGGCATCGGCATCGGAGAGTTC | |
| signal peptide; and | AAGGACAGCCTGAGCATCAACGCCACCAACATCAAGCACT | |
| EGFR cytoplasmic | TCAAGAACTGCACCAGCATCAGCGGCGACCTGCACATTCT | |
| domain and recycling | GCCTGTGGCCTTTAGAGGCGACAGCTTCACCCACACACCT | |
| signal) | CCACTGGATCCCCAAGAGCTGGACATCCTGAAAACCGTGA | |
| | AAGAGATCACCGGATTTCTGTTGATCCAGGCTTGGCCCGA | |
| | GAACCGGACAGATCTGCACGCCTTCGAGAACCTGGAAATC | |
| | ATCAGAGGCCGGACCAAGCAGCACGGCCAGTTTTCTCTGG | |
| | CTGTGGTGTCCCTGAACATCACCAGCCTGGGCCTGAGAAG | |
| | CCTGAAAGAAATCAGCGACGGCGACGTGATCATCTCCGGC | |
| | AACAAGAACCTGTGCTACGCCAACACCATCAACTGGAAGA | |
| | AGCTGTTCGGCACCAGCGGCCAGAAAACAAAGATCATCAG | |
| | CAACCGGGGCGAGAACAGCTGCAAGGCTACAGGCCAAGTG | |
| | TGCCACGCTCTGTGTAGCCCTGAAGGCTGTTGGGGACCCG | |
| | AGCCTAGAGATTGCGTGTCCTGCAGAAACGTGTCCCGGGG | |
| | CAGAGAATGCGTGGACAAGTGCAATCTGCTGGAAGGCGAG | |
| | CCCCGCGAGTTCGTGGAAAACAGCGAGTGCATCCAGTGTC | |
| | ACCCCGAGTGTCTGCCCCAGGCCATGAACATTACCTGTAC | |
| | CGGCAGAGGCCCCGACAACTGTATTCAGTGCGCCCACTAC | |
| | ATCGACGGCCCTCACTGCGTGAAAACATGTCCTGCTGGCG | |
| | TGATGGGAGAGAACAACACCCTCGTGTGGAAGTATGCCGA | |
| | CGCCGGACATGTGTGCCACCTGTGTCACCCTAATTGCACC | |
| | TACGGCTGTACAGGCCCTGGCCTGGAAGGCTGTCCAACAA | |
| | ACGGACCTAAGATCCCCTCTATCGCCACCGGCATGGTTGG | |
| | AGCCCTGCTGCTTCTGCTGGTGGTGGCCCTTGGAATCGGC | |
| | CTGTTTATGCGGCGGAGACACATCGTGCGGAAGGCTAGCT | |
| | ATCAGCCCCTGAGCCAGATCAAGAGACTGCTGAGCGACTC | |
| | CTTCCTGTTCGACAACCCCGTGTACGCTAGCTGA | |
| Modified EGFRt (with | MGAGATGRAMDGPRLLLLLLLGVSLGGARKVCNGIGIGEF | 33 |
| NGFR signal peptide; | KDSLSINATNIKHFKNCTSISGDLHILPVAFRGDSFTHTP | |
| and EGFR cytoplasmic | PLDPQELDILKTVKEITGFLLIQAWPENRTDLHAFENLEI | |
| domain and recycling | IRGRTKQHGQFSLAVVSLNITSLGLRSLKEISDGDVIISG | |
| signal) | NKNLCYANTINWKKLFGTSGQKTKIISNRGENSCKATGQV | |
| | CHALCSPEGCWGPEPRDCVSCRNVSRGRECVDKCNLLEGE | |
| | PREFVENSECIQCHPECLPQAMNITCTGRGPDNCIQCAHY | |
| | IDGPHCVKTCPAGVMGENNTLVWKYADAGHVCHLCHPNCT | |
| | YGCTGPGLEGCPTNGPKIPSIATGMVGALLLLLVVALGIG | |
| | LFMRRRHIVRKASYQPLSQIKRLLSDSFLFDNPVYAS | |
| Nucleotide sequence | ATGCTGCTGCTGGTCACCTCTCTGCTGCTGTGCGAACTGC | 34 |
| encoding modified | CCCATCCTGCCTTTCTGCTGATCCCCAGAAAAGTGTGCAA | |
| EGFRt (with GMS | CGGCATCGGCATCGGAGAGTTCAAGGACAGCCTGAGCATC | |
| SFR alpha signal | AACGCCACCAACATCAAGCACTTCAAGAACTGCACCAGCA | |
| peptide; and NGFR | TCAGCGGCGACCTGCACATTCTGCCTGTGGCCTTTAGAGG | |
| transmembrane and | CGACAGCTTCACCCACACACCTCCACTGGATCCCCAAGAG | |
| cytoplasmic domain | CTGGACATCCTGAAAACCGTGAAAGAGATCACCGGATTTC | |
| and recycling signal) | TGTTGATCCAGGCTTGGCCCGAGAACCGGACAGATCTGCA | |
| | CGCCTTCGAGAACCTGGAAATCATCAGAGGCCGGACCAAG | |
| | CAGCACGGCCAGTTTTCTCTGGCTGTGGTGTCCCTGAACA | |
| | TCACCAGCCTGGGCCTGAGAAGCCTGAAAGAAATCAGCGA | |
| | CGGCGACGTGATCATCTCCGGCAACAAGAACCTGTGCTAC | |
| | GCCAACACCATCAACTGGAAGAAGCTGTTCGGCACCAGCG | |
| | GCCAGAAAACAAAGATCATCAGCAACCGGGGCGAGAACAG | |
| | CTGCAAGGCTACAGGCCAAGTGTGCCACGCTCTGTGTAGC | |
| | CCTGAAGGCTGTTGGGGACCCGAGCCTAGAGATTGCGTGT | |
| | CCTGCAGAAACGTGTCCCGGGGCAGAGAATGCGTGGACAA | |
| | GTGCAATCTGCTGGAAGGCGAGCCCCGCGAGTTCGTGGAA | |
| | AACAGCGAGTGCATCCAGTGTCACCCCGAGTGTCTGCCCC | |
| | AGGCCATGAACATTACCTGTACCGGCAGAGGCCCCGACAA | |
| | CTGTATTCAGTGCGCCCACTACATCGACGGCCCTCACTGC | |
| | GTGAAAACATGTCCTGCTGGCGTGATGGGAGAGAACAACA | |
| | CCCTCGTGTGGAAGTATGCCGACGCCGGACATGTGTGCCA | |
| | CCTGTGTCACCCTAATTGCACCTACGGCTGTACAGGCCCT | |
| | GGCCTGGAAGGCTGTCCAACAAACGGACCTAAGATCCCCT | |
| | CTGGCGGCGGAGGATCTGGCGGAGGTGGAAGCGGAGGCGG | |
| | TGGATCTCTGATCCCCGTGTACTGTAGCATCCTGGCCGCC | |
| | GTGGTTGTGGGACTCGTGGCCTATATCGCCTTCAAGCGGT | |
| | GGAACCGGGGCATCCTGGCTAGCTATCAGCCTCTGAGCCA | |
| | GATCAAGCGGCTGCTGAGCGACTCCTTCCTGTTCGACAAC | |
| | CCTGTGTACGCTAGCTGA | |
| Modified EGFRt (with | MLLLVTSLLLCELPHPAFLLIPRKVCNGIGIGEFKDSLSI | 35 |
| GMS SFR alpha signal | NATNIKHFKNCTSISGDLHILPVAFRGDSFTHTPPLDPQE | |
| peptide; and NGFR | LDILKTVKEITGFLLIQAWPENRTDLHAFENLEIIRGRTK | |
| transmembrane and | QHGQFSLAVVSLNITSLGLRSLKEISDGDVIISGNKNLCY | |
| cytoplasmic domain | ANTINWKKLFGTSGQKTKIISNRGENSCKATGQVCHALCS | |
| and recycling signal) | PEGCWGPEPRDCVSCRNVSRGRECVDKCNLLEGEPREFVE | |
| | NSECIQCHPECLPQAMNITCTGRGPDNCIQCAHYIDGPHC | |
| | VKTCPAGVMGENNTLVWKYADAGHVCHLCHPNCTYGCTGP | |
| | GLEGCPTNGPKIPSGGGGSGGGGSGGGGSLIPVYCSILAA | |
| | VVVGLVAYIAFKRWNRGILASYQPLSQIKRLLSDSFLFDN | |
| | PVYAS | |

In some embodiments the polynucleotide encodes an amino acid sequence having at least 70%, 75%, 80%, 85%, 90%, 95%, 96%, 97%, 98% or 99% identity to SEQ ID NO: 29, 31, 33 or 35.

In some embodiments the polynucleotide encodes the amino acid sequence of SEQ ID NO: 29, 31, 33 or 35.

In some embodiments the polynucleotide comprises a nucleotide sequence having at least 70%, 75%, 80%, 85%, 90%, 95%, 96%, 97%, 98% or 99% identity to SEQ ID NO: 28, 30, 32 or 34.

In some embodiments the polynucleotide comprises a nucleotide sequence of SEQ ID NO: 28, 30, 32 or 34.

In some embodiments the polynucleotide consists of a nucleotide sequence having at least 70%, 75%, 80%, 85%, 90%, 95%, 96%, 97%, 98% or 99% identity to SEQ ID NO: 28, 30, 32 or 34.

In some embodiments the polynucleotide consists of a nucleotide sequence of SEQ ID NO: 28, 30, 32 or 34.

In some embodiments the polynucleotide comprises a nucleotide sequence having at least 70%, 75%, 80%, 85%, 90%, 95%, 96%, 97%, 98% or 99% identity to SEQ ID NO: 37 or 38.

In some embodiments the polynucleotide comprises a nucleotide sequence of SEQ ID NO: 37 or 38.

In some embodiments the polynucleotide consists of a nucleotide sequence having at least 70%, 75%, 80%, 85%, 90%, 95%, 96%, 97%, 98% or 99% identity to SEQ ID NO: 37 or 38.

In some embodiments the polynucleotide consists of a nucleotide sequence of SEQ ID NO: 37 or 38.

In some embodiments the polynucleotide encodes an amino acid sequence having at least 70%, 75%, 80%, 85%, 90%, 95%, 96%, 97%, 98% or 99% identity to SEQ ID NO: 29, 31, 33 or 35, or a fragment thereof.

In some embodiments the polynucleotide encodes the amino acid sequence of SEQ ID NO: 29, 31, 33 or 35, or a fragment thereof.

In some embodiments the polynucleotide comprises a nucleotide sequence having at least 70%, 75%, 80%, 85%, 90%, 95%, 96%, 97%, 98% or 99% identity to SEQ ID NO: 28, 30, 32 or 34, or a fragment thereof.

In some embodiments the polynucleotide comprises a nucleotide sequence of SEQ ID NO: 28, 30, 32 or 34, or a fragment thereof.

In some embodiments the polynucleotide consists of a nucleotide sequence having at least 70%, 75%, 80%, 85%, 90%, 95%, 96%, 97%, 98% or 99% identity to SEQ ID NO: 28, 30, 32 or 34, or a fragment thereof.

In some embodiments the polynucleotide consists of a nucleotide sequence of SEQ ID NO: 28, 30, 32 or 34, or a fragment thereof.

In some embodiments the polynucleotide comprises a nucleotide sequence having at least 70%, 75%, 80%, 85%, 90%, 95%, 96%, 97%, 98% or 99% identity to SEQ ID NO: 37 or 38, or a fragment thereof.

In some embodiments the polynucleotide comprises a nucleotide sequence of SEQ ID NO: 37 or 38, or a fragment thereof.

In some embodiments the polynucleotide consists of a nucleotide sequence having at least 70%, 75%, 80%, 85%, 90%, 95%, 96%, 97%, 98% or 99% identity to SEQ ID NO: 37 or 38, or a fragment thereof.

In some embodiments the polynucleotide consists of a nucleotide sequence of SEQ ID NO: 37 or 38, or a fragment thereof.

In some embodiments the nucleotide sequence encoding the EGFRt is operably linked to a promoter, preferably a weak promoter.

In some embodiments the nucleotide sequence encoding the EGFRt is operably linked to a bidirectional promoter. The bidirectional promoter may be further operably linked to a transgene.

In some embodiments the polynucleotide further comprises an IRES. In some embodiments the nucleotide sequence encoding the EGFRt is downstream of an IRES.

Transgene

In some embodiments the polynucleotide further comprises a transgene. The polynucleotide may be, for example, a bi-cistronic vector or comprise a bi-directional promoter. The bi-cistronic vector may comprise an IRES.

Preferably the transgene gives rise to a therapeutic effect.

In preferred embodiments the polynucleotide comprises a nucleotide sequence encoding a chimeric antigen receptor (CAR).

In some embodiments the polynucleotide comprises a nucleotide sequence encoding a chimeric antigen receptor (CAR), wherein the polynucleotide further comprises a bi-directional promoter. The bi-directional promoter may, for example, control expression of both the transgene and the EGFRt.

In some embodiments the polynucleotide comprises a nucleotide sequence encoding a chimeric antigen receptor (CAR), wherein the polynucleotide is a bi-cistronic vector. The bi-cistronic vector may comprise an IRES.

Chimeric Antigen Receptor (CAR)

CARs comprise an extracellular ligand binding domain, most commonly a single chain variable fragment of a monoclonal antibody (scFv) linked to intracellular signaling components, most commonly CD3 alone or combined with one or more costimulatory domains. A spacer is often added between the extracellular antigen-binding domain and the transmembrane moiety to optimise the interaction with the target.

A CAR for use in the present invention may comprise:
(i) an antigen-specific targeting domain;
(ii) a transmembrane domain;
(iii) optionally at least one costimulatory domain; and
(iv) an intracellular signaling domain.

Preferably, the antigen-specific targeting domain comprises an antibody or fragment thereof, more preferably a single chain variable fragment.

Examples of transmembrane domains include a transmembrane domain of a zeta chain of a T cell receptor complex, CD28 and CD8a.

Examples of costimulatory domains include costimulating domains from CD28, CD137 (4-1BB), CD134 (OX40), DaplO, CD27, CD2, CD5, ICAM-1, LFA-1, Lck, TNFR-I, TNFR-II, Fas, CD30 and CD40.

In some embodiments, the costimulatory domain is a costimulating domain from CD28.

Examples of intracellular signaling domains include human CD3 zeta chain, FcγRIII, FcsRI, a cytoplasmic tail of a Fc receptor and an immunoreceptor tyrosine-based activation motif (ITAM) bearing cytoplasmic receptors.

The term “chimeric antigen receptor” (“CAR” or “CARs”), as used herein, refers to engineered receptors which can confer an antigen specificity onto cells (for example, T cells such as naive T cells, central memory T cells, effector memory T cells or combinations thereof). CARs are also known as artificial T cell receptors, chimeric T cell receptors or chimeric immunoreceptors.

The antigen-specific targeting domain provides the CAR with the ability to bind to the target antigen of interest. The antigen-specific targeting domain preferably targets an antigen of clinical interest against which it would be desirable to trigger an effector immune response.

The antigen-specific targeting domain may be any protein or peptide that possesses the ability to specifically recognise and bind to a biological molecule. The antigen-specific targeting domain includes any naturally occurring, synthetic, semi-synthetic or recombinantly produced binding partner for a biological molecule of interest.

Illustrative antigen-specific targeting domains include antibodies or antibody fragments or derivatives, extracellular domains of receptors, ligands for cell surface molecules/receptors, or receptor binding domains thereof.

In preferred embodiments, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In preferred embodiments, the binding domain is a single chain antibody (scFv). The scFv may be, for example, a murine, human or humanised scFv.

The term “complementarity determining region” (“CDR”) with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

“Heavy chain variable region” (“VH”) refers to the fragment of the heavy chain of an antibody that contains three CDRs interposed between flanking stretches known as framework regions, which are more highly conserved than the CDRs and form a scaffold to support the CDRs.

“Light chain variable region” (“VL”) refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain.

“Single-chain Fv antibody” (“scFv”) refers to an engineered antibody consisting of a light chain variable region and a heavy chain variable region connected to one another directly or via a peptide linker sequence.

Antibodies that specifically bind a target antigen can be prepared using methods well known in the art. Such methods include phage display, methods to generate human or humanised antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

The CAR used in the present invention may also comprise one or more co-stimulatory domains. This domain may enhance cell proliferation, cell survival and development of memory cells.

Each co-stimulatory domain comprises the co-stimulatory domain of any one or more of, for example, members of the TNFR super family, CD28, CD137 (4-1BB), CD134 (OX40), DaplO, CD27, CD2, CD5, ICAM-1, LFA-1, Lck, TNFR-1, TNFR-II, Fas, CD30, CD40 or combinations thereof. Co-stimulatory domains from other proteins may also be used with the CAR used in the present invention.

The CAR used in the present invention may also comprise an intracellular signaling domain. This domain may be cytoplasmic and may transduce the effector function signal and direct the cell to perform its specialised function. Examples of intracellular signaling domains include, but are not limited to, ζ chain of the T cell receptor or any of its homologues (e.g. q chain, FcεR1γ and β chains, MB1 (Igα) chain, B29 (10) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28. The intracellular signaling domain may be human CD3 zeta chain, FcγRIII, FcsRI, cytoplasmic tails of Fc receptors, immunoreceptor tyrosine-based activation motif (ITAM) bearing cytoplasmic receptors or combinations thereof.

The CAR used in the present invention may also comprise a transmembrane domain. The transmembrane domain may comprise the transmembrane sequence from any protein which has a transmembrane domain, including any of the type I, type II or type III transmembrane proteins. The transmembrane domain of the CAR used in the present invention may also comprise an artificial hydrophobic sequence. The transmembrane domains of the CARs used in the present invention may be selected so as not to dimerise. Examples of transmembrane (TM) regions used in CAR constructs are: 1) The CD28 TM region (Pule et al, Mol Ther, 2005, November; 12(5):933-41; Brentjens et al, CCR, 2007, Sep. 15; 13 (18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13 (18 Pt 1):5426-35); 4) The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13 (18 Pt 1):5426-35; Milone et al, Mol Ther, 2009, August; 17(8):1453-64.).

Gene Editing

In some embodiments the polynucleotide, or a part thereof, is for insertion by gene editing.

In some embodiments the polynucleotide is a donor vector or donor template (e.g. in the context of gene editing).

In some embodiments the transgene and/or EGFRt-encoding sequence is for insertion by gene editing.

In some embodiments the polynucleotide further comprises an IRES sequence between the transgene and the nucleotide sequence encoding the EGFRt. In some embodiments the polynucleotide further comprises an IRES sequence between the transgene and the nucleotide sequence encoding the EGFRt, signal peptide and/or the polypeptide (e.g. (i) or (ii), or variant thereof).

In some embodiments the polynucleotide does not comprise a promoter. For example, expression of the EGFRt and/or transgene may be driven by an endogenous promoter in the genome of a cell into which the polynucleotide, or part thereof, is inserted.

For example, the polynucleotide may comprise transgene, IRES and EGFRt-encoding sequences (optionally without a promoter). Such a polynucleotide may be used in, for example, gene editing (e.g. inserted into a cell's genome, optionally wherein expression of the transgene and EGFRt is driven by an endogenous promoter). For example, the gene editing may be applied to cells, such as T cells or hematopoietic stem or progenitor cells.

In a further example context of CD40LG gene editing, the CD40LG gene is generally not expressed in hematopoietic stem or progenitor cells and as a consequence an IRES-EGFRt construct may require a promoter to also be inserted (e.g. a TetO7-minimal promoter upstream of the IRES), either via the polynucleotide of the invention or via a separate system.

The term “gene editing” refers to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations.

Such nucleases may be delivered to a target cell using vectors, such as viral vectors.

Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405; Sander, J. D. et al. (2014) Nat. Biotechnol. 32: 347-55).

Meganucleases (Silve, G. et al. (2011) Cur. Gene Ther. 11: 11-27) may also be employed as suitable nucleases for gene editing.

The CRISPR/Cas system is an RNA-guided DNA binding system (van der Oost et al. (2014) Nat. Rev. Microbiol. 12: 479-92), wherein the guide RNA (gRNA) may be selected to enable a Cas9 domain to be targeted to a specific sequence. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390. [Epub ahead of print]; Zalatan, J. G. et al. (2015) Cell 160: 339-50; Paix, A. et al. (2015) Genetics 201: 47-54), and are suitable for use in the invention.

Polynucleotide

Polynucleotides of the invention may comprise DNA or RNA, preferably DNA. They may be single-stranded or double-stranded. Preferably the polynucleotides are isolated polynucleotides. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Cells

In another aspect the invention provides a cell comprising the polynucleotide of the invention.

In some embodiments the cell is a T cell, lymphocyte or stem cell, such as a hematopoietic stem cell or induced pluripotent stem cell (iPS)

In some embodiments the cell is a haematopoietic stem or progenitor cell. In some embodiments the cell is a T cell.

For example, the cell may be selected from the group consisting of CD4 cells, CD8 cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, double negative T-cells, naive T-cells, memory stem T-cells, central memory T-cells, effector memory T-cells, effector T cells, cytokine-induced killer (CIK) cells, hematopoeitic stem cells and pluripotent stem cells.

The cell may have been isolated from a subject.

The cell of the invention may be provided for use in adoptive cell transfer. As used herein the term “adoptive cell transfer” refers to the administration of a cell population to a patient. Typically, the cells are T cells isolated from a subject and then genetically modified and cultured in vitro before being administered to the patient.

Adoptive cell transfer may be allogenic or autologous.

By “autologous cell transfer” it is to be understood that the starting population of cells (which are then transduced with a polynucleotide or vector according to the invention) is obtained from the same subject as that to which the transduced cell population is administered. Autologous transfer is advantageous as it avoids problems associated with immunological incompatibility and is available to subjects irrespective of the availability of a genetically matched donor.

By “allogeneic cell transfer” it is to be understood that the starting population of cells (which are then transduced with a polynucleotide or vector according to the invention) is obtained from a different subject as that to which the transduced cell population is administered. Preferably, the donor will be genetically matched to the subject to which the cells are administered to minimise the risk of immunological incompatibility. Alternatively, the donor may be mismatched and unrelated to the patient.

Vectors

In some embodiments the polynucleotide is a vector. Preferably the vector is a viral vector, such as a retroviral vector, lentiviral vector, adeno-associated viral (AAV) vector or adenoviral vector. In some embodiments the polynucleotide is a viral genome.

In another aspect the invention provides viral vector comprising the polynucleotide of the invention.

In some embodiments the viral vector is a retroviral vector, lentiviral vector, adeno-associated viral (AAV) vector or adenoviral vector.

In some embodiments the viral vector is in the form of a viral vector particle.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, Pet al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

Adeno-Associated Viral (AAV) Vectors

The AAV vector may comprise an AAV genome or a fragment or derivative thereof.

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain.

In some embodiments the AAV is an AAV1, AAV6, AAV6.2, AAV7, AAV9, rh10, rh39 or rh43 serotype. In some embodiments the AAV vector particle comprises an AAV1, AAV6, AAV6.2, AAV7, AAV9, rh10, rh39 or rh43 serotype capsid protein. In some embodiments, the AAV vector particle is an AAV1, AAV6, AAV6.2, AAV7, AAV9, rh10, rh39 or rh43 vector particle.

In some embodiments, the AAV is an AAV9; AAV9 PHP.B; AAV9 PHP.eB; or AAVrh10 serotype. In some embodiments, the AAV vector particle comprises an AAV9; AAV9 PHP.B; AAV9 PHP.eB; or AAVrh10 serotype capsid protein.

The capsid protein may be an artificial or mutant capsid protein.

The term “artificial capsid” as used herein means that the capsid particle comprises an amino acid sequence which does not occur in nature or which comprises an amino acid sequence which has been engineered (e.g. modified) from a naturally occurring capsid amino acid sequence.

In other words the artificial capsid protein comprises a mutation or a variation in the amino acid sequence compared to the sequence of the parent capsid from which it is derived where the artificial capsid amino acid sequence and the parent capsid amino acid sequences are aligned. Methods of sequence alignment are well known in the art and referenced herein.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the invention on the basis of their common general knowledge.

The AAV serotype determines the tissue specificity of infection (or tropism) of an AAV virus.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments one or more ITR sequences flank the nucleotide sequences disclosed herein. The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

As discussed above, the AAV genome used in the AAV vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

In some embodiments, the AAV vector comprises at least one, such as two, AAV1, 2, 3, 4, 5, 6, 7, 8, 9, 10, or 11 ITRs. In some embodiments, the AAV vector comprises at least one AAV9 ITR.

In some embodiments, the AAV vector comprises two AAV9 ITRs.

The one or more ITRs will preferably flank the nucleotide sequence disclosed herein at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). Thus, in one embodiment the AAV vector is in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

The AAV vector may comprise multiple copies (e.g., 2, 3 etc.) of the nucleotide sequences referred to herein.

Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

Methods of Selection, Depletion and Tracking

In another aspect the invention provides a method of selecting transduced cells comprising the steps:
(a) transducing a population of cells with the polynucleotide or viral vector of the invention;
(b) contacting the transduced cell population with an EGFRt-binding agent; and
(C) selecting the cells bound to the EGFRt-binding agent.

The method of selection may enrich for cells comprising the EGFR epitope or the EGFRt.

In another aspect the invention provides a method of tracking transduced cells comprising the steps:
(a) transducing a population of cells with the polynucleotide or viral vector of the invention;
(b) contacting the transduced cell population with an EGFRt-binding agent, wherein the EGFRt-binding agent is operably linked to a detectable label; and
(c) detecting the cells bound to the EGFRt-binding agent.

Preferably the EGFRt-binding agent binds substantially specifically to EGFRt. In some embodiments the EGFRt-binding agent is an antibody. Agents and antibodies that bind to EGFRt are known in the art and include cetuximab. In some embodiments, the EGFRt-binding agent is cetuximab.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (e.g. the EGFR epitope or the EGFRt of the invention) may be purified from a starting population of cells.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. the EGFR epitope or the EGFRt of the invention) may be achieved by using an agent that binds to that marker, preferably substantially specifically to that marker. An agent that binds to a cellular marker may be an antibody, for example antibody which binds to the EGFR epitope or the EGFRt of the invention.

The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

A number of techniques for detecting (optionally with quantification) a population of cells expressing a specific marker are known in the art. These include flow cytometry, fluorescence-activated cell sorting (FACS), and microscopy-based techniques.

In another aspect the invention provides a method of depleting transduced cells comprising the steps:
(a) transducing a population of cells with the polynucleotide or viral vector of the invention; and
(b) contacting the transduced cell population with an EGFRt-binding agent.

In some embodiments the cell to which the EGFRt-binding agent is bound is killed by antibody-dependent cytotoxicity (ADCC), for example mediated by macrophages.

In some embodiments the EGFRt-binding agent is operably linked to a depletion agent. In some embodiments the cell population of step (b) is contacted with a depletion agent that binds to the EGFRt-binding agent. For example, the EGFRt-binding agent may be operably linked to biotin and the depletion agent may comprise streptavidin, or vice versa.

The depletion agent may kill, preferably selectively kill, a cell to which the EGFRt-binding agent is bound. In some embodiments the depletion agent comprises a toxin. In some embodiments the depletion agent comprises saporin.

In some embodiments the method is an in vitro or ex vivo method.

Method of Treatment

In another aspect the invention provides a polynucleotide, viral vector or cell of the invention for use in therapy.

In another aspect the invention provides a method of treatment comprising the method of selection, depletion or tracking of the invention.

All references herein to treatment include curative, palliative and prophylactic treatment. The treatment of mammals, particularly humans, is preferred. Both human and veterinary treatments are within the scope of the invention.

Pharmaceutical Compositions and Injected Solutions

Although the agents for use in the invention can be administered alone, they will generally be administered in admixture with a pharmaceutical carrier, excipient or diluent, particularly for human therapy.

The medicaments, for example vector particles, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. intravenous or intra-arterial.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

Administration

In some embodiments, the polynucleotide, vector or cell is administered to a subject systemically.

In some embodiments, the polynucleotide, vector or cell is administered to a subject locally.

The term “systemic delivery” or “systemic administration” as used herein means that the agent of the invention is administered into the circulatory system, for example to achieve broad distribution of the agent. In contrast, topical or local administration restricts the delivery of the agent to a localised area.

In some embodiments, the polynucleotide, vector or cell is administered intravascularly, intravenously or intra-arterially.

Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

Variants, Derivatives, Analogues, Homologues and Fragments

In addition to the specific proteins and nucleotides mentioned herein, the invention also encompasses variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Results

In order to evaluate the improvement of EGFRt protein surface expression, we took advantage of a gene editing approach for the correction of the CD40LG gene, mutations in which cause X-linked immunodeficiency with hyper-immunoglobulin M (HIGM1). We integrated into the first intron of the CD40LG gene a corrective construct carrying a codon-optimized version of CD40LG cDNA (from exon 2 to exon 5) coupled to an IRES sequence and an EGFRt (FIG. 1Ai) or modified EGFRt (FIG. 1Aii/iii) sequence, followed by CD40LG endogenous 3′UTR and polyA. With this strategy, CD40LG promoter drives the expression of both CD40LG gene and the EGFRt or modified EGFRt gene. Normally, CD40LG is expressed on the surface of CD4+ T cells only after lymphocyte activation, while at the basal level this protein is not detectable both because of tightly regulated protein translocation and, most importantly, because of a weak CD40LG promoter activation state. We therefore exploited non-activated CD4+ T cells, edited with different corrective constructs carrying the EGFRt or modified EGFRt gene, to study the enhancement of EGFRt stability and surface expression.

We prepared the modified EGFRt sequence by: i) performing the codon optimization of the open reading frame sequence to favour protein translation through the usage of more frequent codons; ii) introducing a novel signal peptide; and iii) introducing a new 8-amino acid cytoplasmic tail taken from either the dNGFR gene (FIG. 1Aii) or from endogenous EGFR gene (FIG. 1Aiii) to anchor and stabilize the protein into the membrane.

Nucleotide sequences of the experimental constructs include:

Unmodified EGFRt
(SEQ ID NO: 36)
TTTACATGTGCTCTTAATTACAGCAGAACCGGTCTGACCTCTT
CTCTTCCTCCCACAGATCGAGGACGAGAGAAACCTGCACGAG
GACTTCGTGTTCATGAAGACCATCCAGCGGTGCAACACCGGC
GGAGAGAAGTCTAGCCTGCTGAACTGCGAGGAAATCAAGAG
CCAGTTCGAGGGCTTCGTGAAGGACATCATGCTGAACAAAGA
GGAAACGAAGAAAGAAAACTCCTTCGAGATGCAGAAGGGCG
ACCAGAATCCTCAGATCGCCGCTCACGTGATCAGCGAGGCCA
GCAGCAAGACAACAAGCGTGCTGCAGTGGGCCGAGAAGGGC
TACTACACCATGAGCAACAACCTGGTCACCCTGGAAAACGGC
AAGCAGCTGACAGTGAAGCGGCAGGGCCTGTACTACATCTAC
GCCCAAGTGACCTTCTGCAGCAACAGAGAGGCCAGCTCTCAG
GCCCCTTTTATCGCCAGCCTGTGCCTGAAGTCCCCTGGCAGAT
TCGAGCGGATTCTGCTGAGAGCCGCCAACACACACAGCAGCG
CCAAACCTTGTGGCCAGCAGTCTATTCACCTCGGCGGAGTGTT
TGAGCTGCAGCCTGGCGCAAGCGTGTTCGTGAATGTGACAGA
CCCTAGCCAGGTGTCCCACGGCACCGGCTTTACATCTTTCGGA
CTGCTGAAGCTGTGAACAGTGTCACCTTGCAGGCTGTGGTGGA
GCTGACGCTGGGAGTCTTCATAATACAGCACAGCGGTTAAGCC
CACCCCCTGTTAACTGCCTATTTATAACCCTAGGGAATTAACTCG
AGGAATTCCGCCCCTCTCCCTCCCCCCCCCCTAACGTTACTGGCCG
AAGCCGCTTGGAATAAGGCCGGTGTGCGTTTGTCTATATGTTATTTT
CCACCATATTGCCGTCTTTTGGCAATGTGAGGGCCCGGAAACCTGG
CCCTGTCTTCTTGACGAGCATTCTAGGGGTCTTTCCCCTCTCGCCA
AAGGAATGCAAGGTCTGTTGAATGTCGTGAAGGAAGCAGTTCCTCT
GGAAGCTTCTTGAAGACAAACAACGTCTGTAGCGACCCTTTGCAGG
CAGCGGAACCCCCCACCTGGCGACAGGTGCCTCTGCGGCCAAAAG
CCAACGTGTATAAGATACACCTGCAAAGGCGGCACAACCCCAGTGC
CACGTTGTGAGTTGGATAGTTGTGGAAAGAGTCAAATGGCTCTCCTC
AAGCGTATTCAACAAGGGGCTGAAGGATGCCCAGAAGGTACCCCAT
TGTATGGGATCTGATCTGGGGCCTCGGTGCACATGCTTTACATGTG
TTTAGTCGAGGTTAAAAAACGTCTAGGCCCCCCGAACCACGGGGAC
GTGGTTTTCCTTTGAAAAACACGATGATAATATGGCCACAACCATGC
TGCTGCTGGTCACCTCTCTGCTGCTGTGCGAACTGCCCCATC
CTGCCTTTCTGCTGATCCCCAGAAAAGTGTGCAACGGCATCG
GCATCGGAGAGTTCAAGGACAGCCTGAGCATCAACGCCACCA
ACATCAAGCACTTCAAGAACTGCACCAGCATCAGCGGCGACC
TGCACATTCTGCCTGTGGCCTTTAGAGGCGACAGCTTCACCC
ACACACCTCCACTGGATCCCCAAGAGCTGGACATCCTGAAAA
CCGTGAAAGAGATCACCGGATTTCTGTTGATCCAGGCTTGGC
CCGAGAACCGGACAGATCTGCACGCCTTCGAGAACCTGGAAA
TCATCAGAGGCCGGACCAAGCAGCACGGCCAGTTTTCTCTGG
CTGTGGTGTCCCTGAACATCACCAGCCTGGGCCTGAGAAGCC
TGAAAGAAATCAGCGACGGCGACGTGATCATCTCCGGCAACA
AGAACCTGTGCTACGCCAACACCATCAACTGGAAGAAGCTGT
TCGGCACCAGCGGCCAGAAAACAAAGATCATCAGCAACCGGG
GCGAGAACAGCTGCAAGGCTACAGGCCAAGTGTGCCACGCTC
TGTGTAGCCCTGAAGGCTGTTGGGGACCCGAGCCTAGAGATT
GCGTGTCCTGCAGAAACGTGTCCCGGGGCAGAGAATGCGTGG
ACAAGTGCAATCTGCTGGAAGGCGAGCCCCGCGAGTTCGTGG
AAAACAGCGAGTGCATCCAGTGTCACCCCGAGTGTCTGCCCC
AGGCCATGAACATTACCTGTACCGGCAGAGGCCCCGACAACT
GTATTCAGTGCGCCCACTACATCGACGGCCCTCACTGCGTGA
AAACATGTCCTGCTGGCGTGATGGGAGAGAACAACACCCTCG
TGTGGAAGTATGCCGACGCCGGACATGTGTGCCACCTGTGTC
ACCCTAATTGCACCTACGGCTGTACAGGCCCTGGCCTGGAAG
GCTGTCCAACAAACGGACCTAAGATCCCCTCTATCGCCACCG
GCATGGTTGGAGCCCTGCTGCTTCTGCTGGTGGTGGCCCTTG
GAATCGGCCTGTTTATGTAGCCTAGGATCCTCCTTATGG
AGAACTATTTATTATACACTCCAAGGCATGTAGAACTGT
AATAAGTGAATTACAGGTCACATGAAACCAAAACGGGC
CCTGCTCCATAAGAGCTTATATATCTGAAGCAGCAACC
CCACTGATGCAGACATCCAGAGAGTCCTATGAAAAGA
CAAGGCCATTATGCACAGGTTGAATTCTGAGTAAACA
GCAGATAACTTGCCAAGTTCAGTTTTGTTTCTTTGCGT
GCAGTGTCTTTCCATGGATAATGCATTTGATTTATCAG
TGAAGATGCAGAAGGGAAATGGGGAGCCTCAGCTCAC
ATTCAGTTATGGTTGACTCTGGGTTCCTATGGCCTTGT
TGGAGGGGGCCAGGCTCTAGAACGTCTAACACAGTGG
AGAACCGAAACCCCCCCCCCCCGCCACCCTCTCGGAC
AGTTATTCATTCTCTTTCAATCTCTCTCTCTCCATCTCT
CTCTTTCAGTCTCTCTCTCTCAACCTCTTTCTTCCAATC
TCTCTTTCTCAATCTCTCTGTTTCCCTTTGTCAGTCTCT
TCCCTCCCCCAGTCTCTCTTCTCAATCCCCCTTTCTAA
CACACACACACACACACACACACACACACACACACAC
ACACACACACACACAGAGTCAGGCCGTTGCTAGTCAG
TTCTCTTCTTTCCACCCTGTCCCTATCTCTACCACTAT
AGATGAGGGTGAGGAGTAGGGAGTGCAGCCCTGAGC
CTGCCCACTCCTCATTACGAAATGACTGTATTTAAAG
GAAATCTATTGTATCTACGATGTCTCCATTGTTTCCAG
AGTGAACTTGTAATTATCTTGTTATTTATTTTTTGAAT
AATAAAGACCTCTTAACATTACGCGCTTAACATTATC
GTTGTTGTTTGAGTACCTAAAGCTCCCAGCCAGGTTG
GGGAAAGAGGAAGCATTTGGAGGGAATTTTCCCAAC
CTTTGTGATGTTTTCATAAACTTTGTTCTCAAGCTACT
TACATT
Key:
SA.CD40LG
IRES
EGFRt with original signal peptide
CD40LG 3′UTR and polyA
Modified EGFRt with NGFR signal peptide and NGFR cytoplasmic domain
(SEQ ID NO: 37)
TTTACATGTGCTCTTAATTACAGCAGAACCGGTCTGACCTCTTC
TCTTCCTCCCACAGATCGAGGACGAGAGAAACCTGCACGAGGA
CTTCGTGTTCATGAAGACCATCCAGCGGTGCAACACCGGCGAG
AGAAGTCTGAGCCTGCTGAACTGCGAGGAAATCAAGAGCCAG
TTCGAGGGCTTCGTGAAGGACATCATGCTGAACAAAGAGGAAA
CGAAGAAAGAAAACTCCTTCGAGATGCAGAAGGGCGACCAGAA
TCCTCAGATCGCCGCTCACGTGATCAGCGAGGCCAGCAGCAAGA
CAACAAGCGTGCTGCAGTGGGCCGAGAAGGGCTACTACACCATG
AGCAACAACCTGGTCACCCTGGAAAACGGCAAGCAGCTGACAGT
GAAGCGGCAGGGCCTGTACTACATCTACGCCCAAGTGACCTTCTG
CAGCAACAGAGAGGCCAGCTCTCAGGCCCCTTTTATCGCCAGCCT
GTGCCTGAAGTCCCCTGGCAGATTCGAGCGGATTCTGCTGAGAGC
CGCCAACACACACAGCAGCGCCAAACCTTGTGGCCAGCAGTCTA
TTCACCTCGGCGGAGTGTTTGAGCTGCAGCCTGGCGCAAGCGTG
TTCGTGAATGTGACAGACCCTAGCCAGGTGTCCCACGGCACCGG
CTTTACATCTTTCGGACTGCTGAAGCTGTGAACAGTGTCACCTTG
CAGGCTGTGGTGGAGCTGACGCTGGGAGTCTTCATAATACAGCA
CAGCGGTTAAGCCCACCCCCTGTTAACTGCCTATTTATAACCCTA
GGGAATTAACTCGAGGAATTCCGCCCCTCTCCCTCCCCCCCCCCTA
ACGTTACTGGCCGAAGCCGCTTGGAATAAGGCCGGTGTGCGTTTGT
CTATATGTTATTTTCCACCATATTGCCGTCTTTTGGCAATGTGAGGGC
CCGGAAACCTGGCCCTGTCTTCTTGACGAGCATTCTAGGGGTCTTT
CCCCTCTCGCCAAAGGAATGCAAGGTCTGTTGAATGTCGTGAAGGA
AGCAGTTCCTCTGGAAGCTTCTTGAAGACAAACAACGTCTGTAGCG
ACCCTTTGCAGGCAGCGGAACCCCCCACCTGGCGACAGGTGCCT
CTGCGGCCAAAAGCCAACGTGTATAAGATACACCTGCAAAGGCGG
CACAACCCCAGTGCCACGTTGTGAGTTGGATAGTTGTGGAAAGAGT
CAAATGGCTCTCCTCAAGCGTATTCAACAAGGGGCTGAAGGATGCC
CAGAAGGTACCCCATTGTATGGGATCTGATCTGGGGCCTCGGTGCA
CATGCTTTACATGTGTTTAGTCGAGGTTAAAAAACGTCTAGGCCCCC
CGAACCACGGGGACGTGGTTTTCCTTTGAAAAACACG

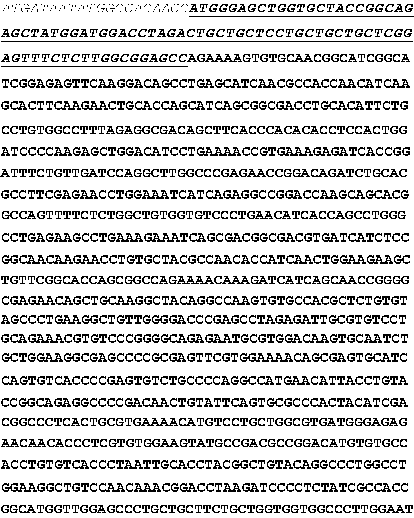

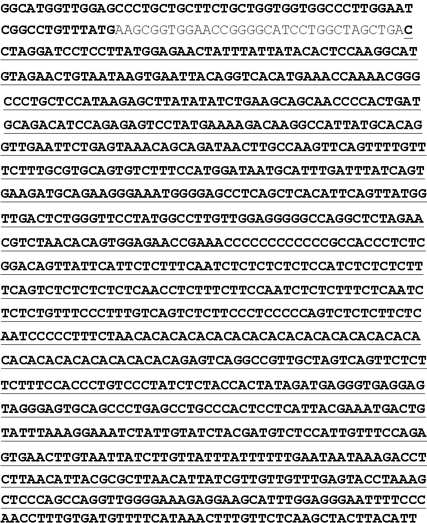

Key:
SA.CD40LG
IRES
dNGFR signal peptide
EGFRt
dNGFR cytoplasmic domain
CD40LG 3′UTR and polyA
Modified EGFRt with NGFR signal peptide and EGFR cytoplasmic domain
(SEQ ID NO: 38)
TTTACATGTGCTCTTAATTACAGCAGAACCGGTCTGACCT
CTTCTCTTCCTCCCACAGATCGAGGACGAGAGAAACCTG
CACGAGGACTTCGTGTTCATGAAGACCATCCAGCGGTGC
AACACCGGCGAGAGAAGTCTGAGCCTGCTGAACTGCGAG
GAAATCAAGAGCCAGTTCGAGGGCTTCGTGAAGGACATC
ATGCTGAACAAAGAGGAAACGAAGAAAGAAAACTCCTTC
GAGATGCAGAAGGGCGACCAGAATCCTCAGATCGCCGCT
CACGTGATCAGCGAGGCCAGCAGCAAGACAACAAGCGTG
CTGCAGTGGGCCGAGAAGGGCTACTACACCATGAGCAAC
AACCTGGTCACCCTGGAAAACGGCAAGCAGCTGACAGTG
AAGCGGCAGGGCCTGTACTACATCTACGCCCAAGTGACC
TTCTGCAGCAACAGAGAGGCCAGCTCTCAGGCCCCTTTTA
TCGCCAGCCTGTGCCTGAAGTCCCCTGGCAGATTCGAGCG GATTCTGCTGAGAGCCGCCAACACACACAGCAGCGCCAA
ACCTTGTGGCCAGCAGTCTATTCACCTCGGCGGAGTGTTT
GAGCTGCAGCCTGGCGCAAGCGTGTTCGTGAATGTGACA
GACCCTAGCCAGGTGTCCCACGGCACCGGCTTTACATCTT
TCGGACTGCTGAAGCTGTGAACAGTGTCACCTTGCAGGCT
GTGGTGGAGCTGACGCTGGGAGTCTTCATAATACAGCACA
GCGGTTAAGCCCACCCCCTGTTAACTGCCTATTTATAACC
CTAGGGAATTAACTCGAGGAATTCCGCCCCTCTCCCTCCCCC
CCCCCTAACGTTACTGGCCGAAGCCGCTTGGAATAAGGCCGG
TGTGCGTTTGTCTATATGTTATTTTCCACCATATTGCCGTCTTTT
GGCAATGTGAGGGCCCGGAAACCTGGCCCTGTCTTCTTGACG
AGCATTCTAGGGGTCTTTCCCCTCTCGCCAAAGGAATGCAAGG
TCTGTTGAATGTCGTGAAGGAAGCAGTTCCTCTGGAAGCTTCT
TGAAGACAAACAACGTCTGTAGCGACCCTTTGCAGGCAGCGG
AACCCCCCACCTGGCGACAGGTGCCTCTGCGGCCAAAAGCCA
ACGTGTATAAGATACACCTGCAAAGGCGGCACAACCCCAGTGC
CACGTTGTGAGTTGGATAGTTGTGGAAAGAGTCAAATGGCTCTC
CTCAAGCGTATTCAACAAGGGGCTGAAGGATGCCCAGAAGGTA
CCCCATTGTATGGGATCTGATCTGGGGCCTCGGTGCACATGCT
TTACATGTGTTTAGTCGAGGTTAAAAAACGTCTAGGCCCCCCGA
ACCACGGGGACGTGGTTTTCCTTTGAAAAACACG
ATGATAATATGGCCACAACCATGGGAGCTGGTGCTACCGGC
AGAGCTATGGATGGACCTAGACTGCTGCTCCTGCTGCTGCTC
GGAGTTTCTCTTGGCGGAGCCAGAAAAGTGTGCAACGGCAT
CGGCATCGGAGAGTTCAAGGACAGCCTGAGCATCAACGCCA
CCAACATCAAGCACTTCAAGAACTGCACCAGCATCAGCGGC
GACCTGCACATTCTGCCTGTGGCCTTTAGAGGCGACAGCTTC
ACCCACACACCTCCACTGGATCCCCAAGAGCTGGACATCCT
GAAAACCGTGAAAGAGATCACCGGATTTCTGTTGATCCAGG
CTTGGCCCGAGAACCGGACAGATCTGCACGCCTTCGAGAAC
CTGGAAATCATCAGAGGCCGGACCAAGCAGCACGGCCAGTT
TTCTCTGGCTGTGGTGTCCCTGAACATCACCAGCCTGGGCCT
GAGAAGCCTGAAAGAAATCAGCGACGGCGACGTGATCATCT
CCGGCAACAAGAACCTGTGCTACGCCAACACCATCAACTGG
AAGAAGCTGTTCGGCACCAGCGGCCAGAAAACAAAGATCAT
CAGCAACCGGGGCGAGAACAGCTGCAAGGCTACAGGCCAAG
TGTGCCACGCTCTGTGTAGCCCTGAAGGCTGTTGGGGACCCG
AGCCTAGAGATTGCGTGTCCTGCAGAAACGTGTCCCGGGGCA
GAGAATGCGTGGACAAGTGCAATCTGCTGGAAGGCGAGCCCC
GCGAGTTCGTGGAAAACAGCGAGTGCATCCAGTGTCACCCCG
AGTGTCTGCCCCAGGCCATGAACATTACCTGTACCGGCAGAG
GCCCCGACAACTGTATTCAGTGCGCCCACTACATCGACGGCC
CTCACTGCGTGAAAACATGTCCTGCTGGCGTGATGGGAGAGA
ACAACACCCTCGTGTGGAAGTATGCCGACGCCGGACATGTGT
GCCACCTGTGTCACCCTAATTGCACCTACGGCTGTACAGGCC
CTGGCCTGGAAGGCTGTCCAACAAACGGACCTAAGATCCCCT
CTATCGCCACCGGCATGGTTGGAGCCCTGCTGCTTCTGCTGGT
GGTGGCCCTTGGAATCGGCCTGTTTATGCGGCGGAGACACAT
CGTGCGGAAGGCTAGCTGACCTAGGATCCTCCTTATGGAGAA
CTATTTATTATACACTCCAAGGCATGTAGAACTGTAATAAGTG
AATTACAGGTCACATGAAACCAAAACGGGCCCTGCTCCATAA
GAGCTTATATATCTGAAGCAGCAACCCCACTGATGCAGACATC
CAGAGAGTCCTATGAAAAGACAAGGCCATTATGCACAGGTTGA
ATTCTGAGTAAACAGCAGATAACTTGCCAAGTTCAGTTTTGTTT
CTTTGCGTGCAGTGTCTTTCCATGGATAATGCATTTGATTTATCA
GTGAAGATGCAGAAGGGAAATGGGGAGCCTCAGCTCACATTCA
GTTATGGTTGACTCTGGGTTCCTATGGCCTTGTTGGAGGGGGCC
AGGCTCTAGAACGTCTAACACAGTGGAGAACCGAAACCCCCCC
CCCCCGCCACCCTCTCGGACAGTTATTCATTCTCTTTCAATCTCT
CTCTCTCCATCTCTCTCTTTCAGTCTCTCTCTCTCAACCTCTTTCT
TCCAATCTCTCTTTCTCAATCTCTCTGTTTCCCTTTGTCAGTCTCT
TCCCTCCCCCAGTCTCTCTTCTCAATCCCCCTTTCTAACACACAC
ACACACACACACACACACACACACACACACACACACACACACA
CAGAGTCAGGCCGTTGCTAGTCAGTTCTCTTCTTTCCACCCTGTC
CCTATCTCTACCACTATAGATGAGGGTGAGGAGTAGGGAGTGCA
GCCCTGAGCCTGCCCACTCCTCATTACGAAATGACTGTATTTAAA
GGAAATCTATTGTATCTACGATGTCTCCATTGTTTCCAGAGTGAA
CTTGTAATTATCTTGTTATTTATTTTTTGAATAATAAAGACCTCTT
AACATTACGCGCTTAACATTATCGTTGTTGTTTGAGTACCTAAAG
CTCCCAGCCAGGTTGGGGAAAGAGGAAGCATTTGGAGGGAATT
TTCCCAACCTTTGTGATGTTTTCATAAACTTTGTTCTCAAGCTACT
TACATT
Key:
SA.CD40LG
IRES
dNGFR signal peptide
EGFRt
EGFR cytoplasmic domain
CD40LG 3′UTR and polyA

We found that only the modified EGFRt (eEGFRt) proteins were measurable on the surface of edited non-activated CD4+ T cells when expressed by the CD40LG endogenous promoter (FIG. 1B), increasing up to 5-fold surface protein expression. Moreover, since eEGFRt proteins were clearly detectable on the CD4+ T cell surface, we were able to enrich targeted cells in vitro (FIG. 10) by exploiting immunomagnetic purification using biotinylated cetuximab.

We also prepared further modified EGFRt sequences that incorporated both dNGFR transmembrane and cytoplasmic domains to the EGFRt extracellular domain through a linker. We tested all the constructs with (FIG. 3) or without recycling signals (FIG. 4), in order to assess boosting the protein recycling on the cell surface. Briefly, we found that the presence of recycling signals in the modified EGFRt did not improve EGFRt expression in respect of the codon optimized sequence (FIG. 3), neither at the basal level or upon T cell activation. However, in absence of any recycling signal, all the modified EGFRt genes improved EGFRt surface expression, showing stable and higher expression (measured as MFI) compared to the codon optimized gene (1) at basal level (FIG. 4).

We then evaluated depletion of eEGFRt+ edited cells in vitro by treating with a specific immunotoxin, composed of streptavidin-saporin (toxin) assembled with biotinylated cetuximab (antibody). With this approach, we observed a reduction of 1.8-fold of eEGFRt+ cells only when cultured in the presence of the assembled immunotoxin, in respect of lymphocytes treated with the toxin alone or the antibody alone (FIG. 1D). Finally, we assessed eEGFRt+ cell depletion in vivo by xenotransplantation of human bulk edited T cells into Nod-Scid-Gamma (NSG) mice. Despite being devoid of T, B and NK cells, these mice retain limited macrophages functionality. Hence, we hypothesized that eEGFRt+ T cells would be depleted (at least partially) by treating mice with by cetuximab, exploiting macrophage-mediated antibody-dependent cytotoxicity (ADCC). Accordingly, we found that eEGFRt-expressing cells were ablated from NSG mice after cetuximab treatment (FIG. 1E).

Thus, by editing cells using modified donor templates, EGFRt protein was expressed on the surface of edited T cells even in absence of stimulation (FIG. 5A) and, importantly, we observed similar gene editing efficiency (FIG. 5B), culture composition (FIG. 5C), CD40L regulated expression (FIG. 5D) and functionality (FIG. 5E,F) as compared to previous templates. In order to evaluate if edited cells were amenable to depletion, we explored a strategy to selectively ablate cells carrying EGFRt with an immunotoxin in vitro. By culturing edited T cells in the presence of Cetuximab conjugated to the protein synthesis inhibitor toxin saporin (Cetuximab-SAP) or of antibody and toxin alone as controls, we observed substantial depletion (˜50%) of EGFRt-expressing lymphocytes at both doses tested (FIG. 5G,H), confirming that the EGFRt is suitable for selection and depletion of edited cells.

In order to confirm the achieved improvement in another gene therapy setting, we took advantage of lentiviral vectors. We cloned two bi-directional lentiviral vectors expressing in sense the GFP reporter gene under the control of a hPGK promoter and in antisense either the tEGFR (FIG. 2Ai) or our modified version (eEGFRt 1—FIG. 2Aii) driven by a minimal CMV promoter. To mimic the context of CAR-T cell therapy, T cells were transduced with these vectors and EGFRt expression was measured. eEGFRt protein surface expression increased up to 39-fold.

In conclusion, we have demonstrated that with the described modifications we were able to improve the use of the EGFRt selector gene even in low-expressing cells, allowing recovery of modified cells in vitro and their depletion in vivo, increasing the safety profile of engineered cells.

Materials and Methods

Primary Cells

Peripheral blood mononuclear cells (PBMCs) were freshly purified from Buffy Coat using Ficoll by sequential centrifugations. Buffy coats were obtained in accordance with the Declaration of Helsinki, as anonymized residues of blood donations, used upon signature of specific institutional informed consents for blood product donation by healthy blood donors. CD4 T cells were isolated by immune-magnetic separation using CD4 T cell isolation kits (Miltenyi Biotech) according the manufacturer's instructions, and stimulated using magnetic beads (ratio cell:bead 1:3) conjugated with anti-CD3/anti-CD28 antibodies (Dynabeads human T-activator CD3/CD28, Thermo Fisher). Cells were maintained in Iscove's modified Dulbecco's medium (IMDM; Corning) supplemented with 10% FBS (Euroclone), penicillin (100 IU/ml), streptomycin (100 μg/ml), 2% glutamine and IL-7 (5 ng/ml; Preprotech) and IL-15 (5 ng/ml; Preprotech). Dynabeads were removed after 6 days of culture. In all experiments T cells were derived from male healthy donors.

T cells were expanded for 21 days to perform flow cytometry and functional analyses and PMA/lonomycin stimulation. When indicated, EGFRt+ edited cells were enriched with anti-biotin microbeads (Miltenyi Biotech), according to the manufacturer's instructions and using a biotinylated Cetuximab antibody (clone #HU1, R&D System). Magnetic separation was performed with LS Columns.

All cells were cultured in a 5% CO₂ humidified atmosphere at 37° C.

Donor Templates and Nucleases

AAV6 donor templates for HDR were generated from a construct containing AAV2 inverted terminal repeats, produced by the TIGEM Vector Core by a triple-transfection method and purified by ultracentrifugation on a cesium chloride gradient. Lentiviral (LV) donor templates for transduction were produced exploiting HIV-derived, third-generation self-inactivating transfer constructs. LV stocks were prepared and titered as previously described (Lombardo, A. et al. (2007) Nat Biotechnol 25: 1298-306).

Ribonucleoproteins (RNPs) were assembled by incubating at 1:2 molar ratio S.p.Cas9 protein (Aldevron) with synthetic cr:tracrRNA (Integrated DNA Technologies) for 10 minutes at 25° C.

Genetic Engineering of Primary Cells

After 3 days of stimulation, gene editing of primary cells was performed. 10⁶ primary T cells were washed with PBS and electroporated with 1.25 μM of ribonucleoprotein (RNP—P3 Primary Cell 4D-Nucleofector X Kit, programs DS-130; Lonza) and transduced with AAV6 15 minutes after electroporation at a dose of 5×10⁴ vg/cell.

After 2 days of stimulation, LV transduction was performed. 10⁶ primary T cells were infected with IDLV at an MOI of 100.

Flow Cytometry Analysis

Cytofluorimetric analyses were performed on FACS Canto II (BD Pharmingen), equipped with DIVA Software and analyzed either with the FSC express software (v. 6, De Novo Software). 7-aminoactinomycin (Sigma Aldrich) was included in the sample preparation for flow cytometry according to the manufacturer's instructions to exclude dead cells from the analysis.

Pma/Ionomyicin Treatment

From 6 to 12 days after electroporation, T cells were stimulated for 5 hours with Phorbol-12-myristate-13-acetate (PMA, 10 ng/ml; Calbiochem) and lonomycin (500 ng/ml, Sigma-Aldrich) in cytokine-free medium, then washed and cultured in complete medium. Surface expression of CD40L on T cells was followed over a 24/48-hour time course by serial flow cytometry analyses (0, 3, 6, 8, 24, 48 hours after activation).

Ex Vivo Functional Studies of Edited T Cells

Naive B cells were isolated from peripheral blood mononuclear cells by immune-magnetic negative selection using the human naive B cell isolation kit II (Miltenyi Biotec), according to the manufacturer's instructions. All cells were cultured in RPMI 1640 (CORNING) containing 1% of penicillin/streptomycin (P/S) (Thermo Fisher scientific), 20% FBS, 20 mM N-2-hydroxyethylpiperazione-N′-2-ethansulfonic acid (HEPES) (both from Sigma-Aldrich), 1% L-glutamine (Life Technologies) and 55 uM 2-mercaptoethanol (Gibco—Life Technologies).

Prior to co-culture, CD4 T cells were washed and rested overnight in cytokine-free medium. T cells were then activated for 5 hours using two different stimuli:
1. CD3/CD28 Dynabeads (Gibco—Life Technologies) at a 1:1 bead:cell ratio;
2. phorbol myristate acetate (PMA 1 ng/mL, Sigma Aldrich) plus ionomycin (500 ng/mL, Sigma Aldrich).

After removing Dynabeads and PMA/lonomycin, and washing cells with complete medium, B and T cells were co-cultured in 200 uL of medium previously described at a 1:3 B-cell:T-cell ratio in a 96 well plates flat bottom (CORNING).

T and B cell cells were kept in culture for 5 days in the presence of IL-2 (50 ng/mL), IL-7 (5 ng/mL) and IL-15 (5 ng/mL) (all form PeproTech) and B cells were stimulated using various combination of the following cytokines: IL-21 (100 ng/mL) (PeproTech), human Toll-like receptor 9 (TLR9) agonist CpG oligodeoxinucleotide (ODN) 1826 (2.5 ug/mL) (InvivoGen), anti-IgA+ IgG+ IgM (15 ug/mL, goat anti-human IgA+ IgG+ IgM) (Jackson ImmunoResearch) and soluble CD40L (3 ug/mL) (ENZO Life Sciences).

To evaluate B cell proliferation, naive B cells were labeled with CellTrace™ Violet Cell Proliferation Kit (ThermoFisher Scientific) following the manufacturer's instructions and then co-cultured with T cells following the protocol previously described. The proliferation was analyzed after 5 days of T/B co-culture by FACS.

Immunoglobulin-secreting cells were analyzed 5 days after co-culture by ELISPOT assay performed in plates with nitrocellulose membrane (Merk Millipore) coated with anti-IgG or anti-IgM (both from Southern Biotech). After blocking with PBS (CORNING) and 1% BSA (Sigma-Aldrich), serial dilution of total cells (from 0.5×10⁴ to 0.25×10⁴) were added and incubated overnight at 37° C. Plates were then incubated with isotype-specific secondary antibodies (both from Southern Biotech), followed by streptavidin-HRP (ThermoFisher) and finally developed with 3-amino-9-ethylcarbazole (Sigma Aldrich) as a chromogenic substrate. Plates were scanned and counted using the Automated ELISA-Spot Assay Video Analysis System (AELVIS) to determine the number of spots/wells.

Molecular Analysis

For digital droplet PCR (ddPCR) analysis, 5-50 ng of genomic DNA were analyzed using the QX200 Droplet Digital PCR System (Biorad) according to the manufacturer's instructions. HDR ddPCR primers and probe were designed on the junction between the donor sequence and the targeted locus and on control sequences used for normalization (human TTC5 gene, PrimePCR ddPCR Copy Number Assay, Biorad). Thermal conditions for annealing and extension were adjusted as following: 55° C. for 1 minute, 72° C. for 2 minutes.

In Vitro Depletion

For in vitro depletion, 5000 T cells/well were seeded in complete medium in 96-well round bottom plates. The αEGFR-SAP immunotoxin was prepared by combining biotinylated Cetuximab antibody (clone #Hu1, R&D Systems) with streptavidin-SAP conjugate (2.3 saporin molecules per streptavidin, Advanced Targeting Systems) in a 1:1 molar ratio and diluted in PBS at two doses (5 nM, 1 nM) immediately before use. Immunotoxin, and the same quantity of antibody alone or toxin alone were added to cells for three days and then lymphocytes were collected for flow cytometry analysis.

In Vivo Depletion

At day 20 of culture, 10×10⁶ bulk edited CD4 T cells were injected intravenously into 7 to 10-weeks old male NSG mice. Sample size was determined by the total counts of available treated cells. Two weeks after transplantation, half of experimental mice were treated with 1 mg/mouse of Cetuximab (Erbitux; Merck, 5 mg/ml) for 4 days, by intraperitoneal injections. Attribution of mice to each experimental group was random. The presence of gene-edited cells (by FACS and by ddPCR) was monitored by serial collection of blood from the mouse eye. Mice were weekly monitored and weighed to eventually observe appearance of signs of graft versus host disease.

All publications mentioned in the above specification are herein incorporated by reference. Various modifications and variations of the disclosed polynucleotides, proteins, vectors, cells, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A polynucleotide comprising a nucleotide sequence encoding an epidermal growth factor receptor (EGFR) extracellular epitope operably linked to:
(a) a NGFR or GMS SFR alpha signal peptide;
(b) a EGFR or NGFR transmembrane domain; and/or
(c) a NGFR or EGFR cytosplasmic tail, optionally wherein the cytoplasmic tail comprises an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions,
and optionally a recycling signal, and further optionally wherein the EGFR extracellular epitope is a truncated epidermal growth factor receptor (EGFRt).

2. A polynucleotide comprising a nucleotide sequence encoding a truncated epidermal growth factor receptor (EGFRt), wherein the polynucleotide further comprises:
(a) a nucleotide sequence encoding a NGFR signal peptide; and/or
(b) a nucleotide sequence encoding a polypeptide comprising an amino acid sequence of: (i) KRWNRGIL (SEQ ID NO: 39); or (ii) RRRHIVRK (SEQ ID NO: 40); or a variant of (i) or (ii) having up to three amino acid substitutions, additions or deletions.

3. The polynucleotide of claim 1 or 2, wherein:
(a) the NGFR signal peptide comprises an amino acid sequence having at least 70% identity to SEQ ID NO: 4 or a fragment thereof; and/or
(b) the nucleotide sequence encoding the NGFR signal peptide has at least 70% identity to SEQ ID NO: 5 or a fragment thereof.

4. The polynucleotide of any preceding claim, wherein the EGFRt comprises a EGFR Domain III, a EGFR Domain IV and a EGFR transmembrane domain.

5. The polynucleotide of any preceding claim, wherein the EGFRt does not comprise a EGFR Domain I, a EGFR Domain II, a EGFR Juxtamembrane Domain or a EGFR Tyrosine Kinase Domain.

6. The polynucleotide of any preceding claim, wherein the EGFRt comprises an amino acid sequence having at least 70% identity to SEQ ID NO: 2 or a fragment thereof.

7. The polynucleotide of any preceding claim, wherein the nucleotide sequence encoding the EGFRt has at least 70% identity to SEQ ID NO: 3 or a fragment thereof.

8. The polynucleotide of any preceding claim, wherein the polynucleotide:
(a) encodes an amino acid sequence having at least 70% identity to SEQ ID NO: 17 or a fragment thereof; and/or
(b) comprises a nucleotide sequence having at least 70% identity to SEQ ID NO: 16 or a fragment thereof.

9. The polynucleotide of any preceding claim, wherein the polynucleotide:
(a) encodes an amino acid sequence having at least 70% identity to SEQ ID NO: 19 or 21, or a fragment thereof; and/or
(b) comprises a nucleotide sequence having at least 70% identity to SEQ ID NO: 18 or 20, or a fragment thereof.

10. The polynucleotide of any preceding claim, wherein the polynucleotide:
(a) encodes an amino acid sequence having at least 70% identity to SEQ ID NO: 23 or 25, or a fragment thereof; and/or
(b) comprises a nucleotide sequence having at least 70% identity to SEQ ID NO: 22 or 24, or a fragment thereof.

11. The polynucleotide of any preceding claim, wherein the nucleotide sequence encoding the EGFRt is operably linked to a weak promoter.

12. The polynucleotide of any preceding claim, wherein the polynucleotide further comprises a transgene, optionally wherein an IRES element is between the transgene and the nucleotide sequence encoding the EGFRt.

13. The polynucleotide of any preceding claim, wherein the polynucleotide, for example comprising the transgene and/or EGFRt-encoding sequence, is for insertion by gene editing.

14. The polynucleotide of claim 12, wherein the transgene encodes a chimeric antigen receptor.

15. An EGFRt protein encoded by the polynucleotide of any preceding claim.

16. A viral vector comprising the polynucleotide of any one of claims 1-14.

17. The viral vector of claim 16, wherein the viral vector is a lentiviral vector, adeno-associated viral (AAV) vector or adenoviral vector.

18. A cell comprising the polynucleotide of any one of claims 1-14 or the viral vector of claim 16 or 17.

19. The polynucleotide, viral vector or cell of any one of claims 1-14 or 16-18 for use in therapy.

20. A method of selecting transduced cells comprising the steps:
(a) transducing a population of cells with the polynucleotide or viral vector of any one of claims 1-14, 16 or 17;
(b) contacting the transduced cell population with an EGFRt-binding agent; and
(c) selecting the cells bound to the EGFRt-binding agent.

21. A method of depleting transduced cells comprising the steps:
(a) transducing a population of cells with the polynucleotide or viral vector of any one of claims 1-14, 16 or 17; and
(b) contacting the transduced cell population with an EGFRt-binding agent,
optionally wherein (i) the EGFRt-binding agent is operably linked to a depletion agent, or (ii) the cell population of step (b) is contacted with a depletion agent that binds to the EGFRt-binding agent, wherein the depletion agent kills a cell to which the EGFRt-binding agent is bound.

22. A method of tracking transduced cells comprising the steps:
(a) transducing a population of cells with the polynucleotide or viral vector of any one of claims 1-14, 16 or 17;
(b) contacting the transduced cell population with an EGFRt-binding agent, wherein the EGFRt-binding agent is operably linked to a detectable label; and
(c) detecting the cells bound to the EGFRt-binding agent.

23. The method of any one of claims 20-22, wherein the method is an in vitro or ex vivo method.

24. The method of any one of claims 20-23, wherein the EGFRt-binding agent is an antibody, optionally wherein the EGFRt-binding agent is cetuximab

25. The method of any one of claim 21, 23 or 24, wherein the depletion agent comprises a toxin, optionally wherein the depletion agent comprises saporin.

26. A method of treatment comprising the method of any one of claims 21-25.
